(19) United States
(12) Patent Application Publication
(10) Pub. No.: US 2005/0100887 A1
(43) Pub. Date: May 12, 2005

(54) Targets for hepatitis C virus infections

(76) Inventors: Konstadinos Salassidis, Eching (DE); Daniel Schubart, Berlin (DE); Heidrun Gutbrod, Graefelfing (DE); Stefan Mueller, Munich (DE); Friedrich Kraetzer, Munich (DE); Sabine Obert, Munich (DE)

Correspondence Address: LEON R. YANKWICH;YANKWICH & ASSOCIATES, 201 BROADWAY, CAMBRIDGE, MA, 02139, US

(21) Appl. No.: 10/872,645

(22) Filed: Jun. 21, 2004

Related U.S. Application Data
(63) Continuation-in-part of application No. PCT/EP02/14578, filed on Dec. 19, 2002.
(60) Provisional application No. 60/341,757, filed on Dec. 21, 2001.

(51) Int. Cl. C12Q 1/70; A61K 38/12; A61K 38/05

(52) U.S. Cl. 435/5; 514/9; 514/18

(57) ABSTRACT

Methods for the detection of compounds useful for prophylaxis and/or treatment of Hepatitis C virus infections and methods for detecting Hepatitis C virus infections in an individual or in cells are disclosed. Mono- or polyclonal antibodies are disclosed effective for the treatment of HCV infections together with methods for treating Hepatitis C virus infections or for the regulation of Hepatitis C virus production and/or replication wherein said antibodies are disclosed. Solid supports useful for detecting Hepatitis C virus infections or for screening compounds useful for prophylaxis and/or treatment of HCV infections are also disclosed.

### CROSS-REFERENCE TO RELATED APPLICATIONS

This application is a continuation-in-part of pending international application PCT/EP02/14578, filed Dec. 19, 2002, designating the United States, which claims priority to U.S. provisional application Ser. No. 60/341,757, filed Dec. 21, 2001.

### FIELD OF THE INVENTION

The present invention relates to human cellular protein kinases, metalloproteases and phosphatases as potential targets for medical intervention against Hepatitis C virus (HCV) infections. Particularly, the present invention relates to a method for the detection of compounds useful for prophylaxis and/or treatment of Hepatitis C virus infections and a method for detecting Hepatitis C virus infections in an individual or in cells. Also mono- or polyclonal antibodies are disclosed effective for the treatment of HCV infections together with methods for treating Hepatitis C virus infections or for the regulation of Hepatitis C virus production wherein said antibodies may be used. Finally the present invention relates to a solid support useful for detecting Hepatitis C virus infections or for screening compounds useful for prophylaxis and/or treatment of HCV infections.

### BACKGROUND OF THE INVENTION

Hepatitis C Virus (HCV) infection is a major cause of chronic hepatitis, cirrhosis and hepatocellular carcinoma. The WHO estimates that approximately 3% of the world population, or 170 million people, have been infected with the Hepatitis C Virus. In the U.S., an estimated 3.9 million Americans have been infected (CDC fact sheet September 2000). Over 80% of HCV-infected individuals develop chronic hepatitis, which is associated with disease states ranging from asymptomatic carrier states to repeated inflammation of the liver and serious chronic liver disease. Over the course of 20 years, more than 20% of chronic HCV-patients are expected to be at risk to develop cirrhosis or progress to hepatocellular carcinoma. Liver failure from chronic hepatitis C is the leading indicator for liver transplantation. Excluding transplantation, the CDC estimates that medical and work-loss cost for HCV annually are around $600 million. HCV is transmitted primarily by blood and blood products. Due to routine screening of the blood supplies from mid-1992, new transfusion-related cases are exceedingly rare and have been surpassed by injection drug use as the highest risk factor for acquiring the virus. There is also a sexual, however inefficient, route of transmission, and a 6% rate of transmission from infected mothers to their children, which is higher in case of HIV coinfection. In a certain percentage of infections, the mode of transmission remains unknown. In spite of the significant decline in incidence in the 1990's, the number of deaths (estimated deaths annually at the moment: 8000 to 10,000 in U.S.) and severe disease due to HCV is anticipated to triple in the next 10 to 20 years. (Sources: CDC fact sheet (accessed Dec. 12, 2000); Houghton M. Hepatitis C Viruses. In B N Fields, D M Knipe, P M Howley (ed.) Fields Virology. 1996. Lippencott-Raven Pub., Philadelphia; Rosen H R and Gretch D R, Molecular Medicine Today Vol5, 393, September 1999; Science 285, 26, July 1999: News Focus: The scientific challenge of Hepatitis C; Wong J B et al, Am J Public Health, 90, 1562, October 2000: Estimating future hepatitis C morbidity, mortality, and costs in the United States).

According to the Consensus Statement from the EASL (European Association for the Study of the Liver) International Consensus Conference on Hepatitis C (Feb. 26-28, 1999, Paris, France), combination therapy of alpha interferon and ribavirin is the recommended treatment for naive patients. Monotherapy with interferon has also been approved by the FDA, but the sustained response rate (HCV RNA remains undetectable in the serum for more than 6 months after end of therapy) is only 15 to 20%, in contrast to 35 to 45% with combination therapy. Interferons (Intron A, Schering-Plough; Roferon A, Hoffmann-LaRoche; Wellferon, Glaxo Wellcome; Infergen, Amgen) are injected subcutaneously three times a week, ribavirin (Rebetol, Schering-Plough) is an oral drug given twice a day. Recommended treatment duration is 6 to 12 months, depending on HCV genotype. Experimental forms of slow-release pegylated interferons (Pegasys, Hoffmann-LaRoche; PEG-Intron, Schering-Plough) have shown improvements in response rates (42 to 82% in combination with ribavirin) and application (once-weekly injection) in recent clinical studies (Hepatology32:4, Pt 2 of 2. October 2000; NEJM 343, 1673. December 2000; NEJM 343, 1666. December 2000). Common side effects of interferon therapy include: fatigue, muscle aches, head aches, nausea, fever, weight loss, irritability, depression, bone marrow suppression, reversible hair loss. The most common side effects of ribavirin are anemia, fatigue and irritability, itching, skin rash, nasal stuffiness, sinusitis, cough. More serious side effects of mono-and combination therapy occur in less than two percent of patients (NIDDK information: Chronic Hepatitis C: Current Disease Management. accessed Sep. 12, 1999). Some of the contraindications to interferon are psychosis or severe depression; neutropenia and/or thrombocytopenia; organ transplantation except liver; symptomatic heart disease; decompensated cirrhosis; uncontrolled seizures. Contraindications to ribavirin are end-stage renal failure; anemia; hemoglobinopathies; severe heart disease; pregnancy; no reliable method of contraception (consensus statement EASL).

Experimental treatments that are not new forms of interferon are Maxamine (histamine dihydrochloride, Maxim Pharmaceuticals), which will be combined with Interferon in phase III studies, VX-497 (Vertex Pharmaceuticals), an IMP dehydrogenase inhibitor, as a less toxic ribavirin substitute in phase II, and amantadine (Endo Labs), an approved influenza drug, as the third component in triple therapy (phase II). Inhibitors for HCV enzymes such as protease inhibitors, RNA polymerase inhibitors, helicase inhibitors as well as ribozymes and antisense RNAs are under preclinical development (Boehringer Ingelheim, Ribozyme Pharmaceuticals, Vertex Pharmaceuticals, Schering-Plough, Hoffmann-LaRoche, Immusol, Merck etc.). No vaccine is available for prevention or therapeutic use, but several companies are trying to develop conventional or DNA vaccines or immunostimulatory agents (e.g. Chiron, Merck/Vical, Epimmune, NABI, Innogenetics).

In summary, the available treatment for chronic Hepatitis C is expensive, effective only in a certain percentage of patients, and adverse side effects are not uncommon.

### DESCRIPTION OF THE INVENTION

Recent research has revealed how cells communicate with each other to coordinate the growth and maintenance of the multitude of tissues within the human body. A key element of this communication network is the transmission of a signal from the exterior of a cell to its nucleus, which results in the activation or suppression of specific genes. This process is called signal transduction.

An integral part of signal transduction is the interaction of ligands, their receptors and intracellular signal transduction molecules. Ligands are messengers that bind to specific receptors on the surface of target cells. As a result of the binding, the receptors trigger the activation of a cascade of downstream signaling molecules, thereby transmitting the message from the exterior of the cell to its nucleus. When the message reaches the nucleus, it initiates the modulation of specific genes, resulting in the production of RNA and finally proteins that carry out a specific biological function. Disturbed activity of signal transduction molecules may lead to the malfunctioning of cells and disease processes. Specifically, interaction of HCV with host cells is necessary for the virus to replicate.

The present invention is based upon the discovery of a group of human cellular protein kinases, metalloproteases and one phosphatase which are specifically up- or downregulated as a result of HCV replication in HCV infected host cells. The antiviral therapeutic research approach described herein focuses on discovering the cellular signal transduction pathways involved in viral infections. Identification of the signal transduction molecules, key to viral infection, provides for, among other things, novel diagnostic methods, for example, assays and compositions useful therefore, novel targets for antiviral therapeutics, a novel class of antiviral therapeutics, and new screening methods (e.g. assays) and materials to discover new antiviral agents.

This approach led to the development of a novel microarray platform technology, wherein a microarray of more than 900 signal transduction cDNAs was developed. This unique microarray technology was used to identify changes in RNA expression patterns (e.g. upregulation or downregulation) as a result of HCV infected host cells. Differential display techniques were used to pinpoint those signal transduction molecules useful as targets for drug intervention. Effective manipulation of these virally-controlled intracellular signal transduction pathways can alter (slow or stop altogether) the course of viral growth.

It is an object of the present invention to provide novel targets for medical intervention, prophylaxis and/or treatment of Hepatitis C virus infections in mammals, including humans, and cells together with methods for detecting HCV infections in individuals and cells and methods for detecting compounds useful for prophylaxis and/or treatment of HCV infections. The object of the present invention is solved by the teachings herein.

It is now revealed for the first time that the human cellular proteins:
(1) beta-adrenergic receptor kinase 1 (gene accession number NM_{—}001619, other names: X61157, ADRBK1, GRK2, BARK1), (amino acid sequence SEQ ID NO: 24, DNA Sequence SEQ ID NO: 25);
(2) Mitogen activated protein kinase activated protein kinase 5 (also known as: AF032437, MAPKAPK5, or PRAK), (amino acid sequence SEQ ID NO: 1, DNA sequence SEQ ID NO: 2);
(3) Insulin-stimulated protein kinase 1 (also known as: U08316, ribosomal protein S6 kinase 3 90K), (amino acid sequence SEQ ID NO: 3, DNA sequence SEQ ID NO: 4);
(4) Discoidin domain receptor family, member 1 (gene accession number NM_{—}013994, other names: X74979, DDR1, TRK E, NEP, or CAK), (amino acid sequence SEQ ID NO: 27, DNA Sequence SEQ ID NO: 28);
(5) Protein Kinase C, mu (also known as: X75756 or PKC-mu), (amino acid sequence SEQ ID NO: 5, DNA sequence SEQ ID NO: 6);
(6) Protein Kinase C, theta (also known as: L01087 or PKC-theta), (amino acid sequence SEQ ID NO: 7, DNA sequence SEQ ID NO: 8);
(7) AMP-activated protein kinase beta 2 subunit (also known as: AJ 224538 or AMPK beta 2), (amino acid sequence SEQ ID NO: 9, DNA sequence SEQ ID NO: 10);
(8) JNK2 (also known as: U09759 or L31951), (amino acid sequence SEQ ID NO: 11, DNA sequence SEQ ID NO: 12);
(9) Human p21-activated protein kinase 2 (also known as: U24153 or PAK2), (amino acid sequence SEQ ID NO: 13, DNA sequence SEQ ID NO: 14);
(10) cyclin-dependent kinase 4 (also known as: U37022 or cdk4), (amino acid sequence SEQ ID NO: 15, DNA sequence SEQ ID NO: 16);
(11) MEK5 (also known as: U25265 or Mitogen-activated protein kinase kinase 5), (amino acid sequence SEQ ID NO: 17, DNA sequence SEQ ID NO: 18);
(12) MKP-L (gene accession number: NM_{—}007026; also known as AF038844), (amino acid sequence SEQ ID NO: 26, DNA sequence SEQ ID NO: 23);
(13) ADAM22 (gene accession number: NM_{—}016351, a disintegrin and metalloproteinase domain 22, also known as AF155382) (amino acid sequence SEQ ID NO: 19, DNA sequence SEQ ID NO: 20); and
(14) ADAM17 (also known as: U92649 or a disintegrin and metalloproteinase domain 17 (tumor necrosis factor, alpha, converting enzyme) also known as XM_{—}002270) (amino acid sequence SEQ ID NO: 21, DNA sequence SEQ ID NO: 22); are specifically and uniquely up- or downregulated in a cell as a result of HCV infection. These cellular protein kinases, metalloproteases and the phosphatase therefore identify novel diagnostic and therapeutic targets for HCV infection.

The only reliable experimental HCV infection studies have been performed with chimpanzees. There is no simple cell culture infection system available for HCV. Although a number of reports have been published describing in vitro propagation attempts of HCV in primary cells and cell lines, questions remain concerning reproducibility, low levels of expression and properly controlled detection methods (reviewed in J. Gen Virol. 81, 1631; Antiviral Chemistry and Chemotherapy 10, 99). Thus, the replicon system described by Bartenschlager and coworkers (Lohmann et al, Replication of subgenomic hepatitis C virus RNAs in a hepatoma cell line. Science 285, 110. 1999) was used for the studies disclosed herein. This replicon system reproduces a crucial part of the HCV replication cycle which is used as a system for simulating HCV infection. Bartenschlager's group produced bicistronic recombinant RNAs, so-called “replicons”, which carry the Neomycin-phosphotransferase gene as well as a version of the HCV genome where the sequences for the structural HCV proteins were deleted. After transfection of the subgenomic HCV RNA molecules into the human hepatoma cell line Huh-7, cells supporting efficient RNA-dependent RNA replication of the HCV replicons were selected based on co-amplification of the neo gene and resulting resistance to the antibiotic G418. Integration of coding information into the cellular genome was an exclusion criterium for functional replicons. Several lines were established from G418 resistant clones with autonomously replicating HCV RNAs detectable by Northern blot. Minus-strand RNA replication intermediates were detected by Northern blot or metabolic radio-labeling, and the production of nonstructural HCV proteins was demonstrated by immuno-precipitation after metabolic labeling or Western blot.

Possible influences and/or dependencies of HCVs RNA-dependent RNA replication and nonstructural proteins on host cell transcription are accessible to analysis with the cDNA arrays used in the inventive methods described herein. Expression levels can be confirmed using Northern or Taqman analysis at the RNA and Western blot analysis at the protein level. Huh-pcDNA3 cells are Huh7 cells resistant to G418 by integration of a plasmid and serve as negative control. Three replicon lines were analyzed for changes in cellular RNA expression patterns compared to the control line:
Huh-9-13: cell line with persistant replicon I377/NS3-3′/wt, described in Science 1999, 285, 110-113;
Huh-5-15: cell line with persistant replicon I389/NS3-3′/wt, described in Science 1999, 285, 110-113; and
Huh-11-7: cell line with persistant replicon I377/NS2-3′/wt, described in Science 1999, 285, 110-113.

Based on the discoveries reported herein, one aspect of the present invention is directed to a screening method for detecting compounds useful for the prophylaxis and/or treatment of Hepatitis C virus infections. Specifically, this method involves contacting a test compound with at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta(L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (AF038844, NM_{—}007026), ADAM22 (NM-_{—}016351), ADAM17 (U92649) and detecting the human cellular protein kinase, metalloprotease or phosphatase activity.

Another aspect of the present invention is directed to a diagnostic method, an assay for detecting Hepatitis C virus infections in an individual or cells. This method involves providing a sample from the individual or providing cells and detecting activity of at least one human cellular protein kinase, metalloprotease or phosphatase selected from the group consisting of: beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), ADAM17 (U92649) and detecting the human cellular protein kinase, metalloprotease or phosphatase activity.

Accordingly, one aspect of the present invention is directed to novel compounds useful in the above-identified methods. Therefore, the present invention relates to a monoclonal or polyclonal antibody that binds to a human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM-007026), ADAM22 (NM_{—}016351) and ADAM17 (U92649).

Furthermore, the present invention discloses a method for treating Hepatitis C virus infection in an individual comprising the step of administering a pharmaceutically effective amount of an agent which inhibits at least partially the activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

Another object of the present invention is to provide a method for regulating the production of Hepatitis C virus in cells comprising the step of administering a pharmaceutically effective amount of an agent to said cells wherein said agent inhibits at least partially the activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649). The above-mentioned monoclonal or polyclonal antibodies directed against these targets may be used as pharmaceutically active agents within said methods.

In order to identify HCV infections and new inhibitors and new pharmaceutically active compounds against Hepatitis C viruses a further aspect of the present invention is directed to a solid support useful for detecting Hepatitis C virus infections in an individual or in cells comprising an immobilized oligonucleotide, wherein said oligonucleotide is capable of detecting activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649). Said solid support is also useful to screen compounds for the prophylaxis and/or treatment of Hepatitis C virus infections in an individual comprising at least one immobilized oligonucleotide, wherein said oligonucleotide encodes one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649) or comprising at least one immobilized human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

Yet another aspect of the present invention is directed to a novel therapeutic composition useful for the prophylaxis and/or treatment of an individual afflicted with Hepatitis C virus comprising at least one agent capable of inhibiting activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

### BRIEF DESCRIPTION OF THE DRAWINGS

FIG. 1A is a chart illustrating the GRK2 mRNA expression levels, showing that GRK2 mRNA expression was increase in 4 different subgenomic HCV replicon lines compared to the control cell line Huh-pc. Total RNA was isolated from subconfluent HCV replicon lines Huh-5-15, 5-2, 9-13, 11-7 and the control cell line Huh-pcDNA3 (Huh-pc). Reverse transcription products were quantitated by GRK2-specific quantitative real-time PCR analysis. Expression levels are given relative to the GRK2 levels of the control cell line.

FIG. 1B is a table showing the relative expression levels of GRK2 mRNA as determined using the procedure from FIG. 1A at three independent time points. The notation “nd” means a determination was not done at that particular time point.

FIG. 2A is photograph of a gel showing that reduction of GRK2 level correlated with a reduction of HCV RNA replication in a subgenomic replicon line. Huh-5-2 replicon cell were transfected with 50 nM of sequence-specific siRNAs targeting GRK2 (siRNA-A, siRNA-C), the luciferase gene encoded by the 5-2 replicon (siRNA-Luc) or a control siRNA (GL3). Cells were lysed on day 3 after one transfection (1st transfection) or two subsequent transfections (2nd transfection, see methods). IFN, samples from cells treated with 100 units/ml of interferon alpha. Lysates were separated on SDS-PAGE and analyzed by Western blotting with anti-GRK2 (upper panel) or anti-tubulin antibody (lower panel, loading control).

FIG. 2B is a chart showing luciferase activity as a measure of HCV subgenomic replicon content. Aliquots of the cell lysates were assayed for luciferase activity as a measure of HCV subgenomic replicon content. The activity of the control sample (GL3) was arbitrarily set to 100%.

### DETAILED DESCRIPTION OF THE INVENTION

Utilizing microarray technology, a unique microarray of more than 900 signal transduction cDNAs was developed. This array was used to compare signal transduction mRNA expression patterns (e.g. upregulation or downregulation) from HCV Replicon cells Huh-9-13, Huh-5-15, and Huh-11-7 with Huh-pcDNA control cells which do not contain HCV Replicons. These HCV Replicon cells serve as a system for simulation of HCV infected cell systems, especially for simulating HCV infected mammals, including humans. Interference of HCV with the cellular signaling events is reflected in differential gene expression when compared to cellular signaling in control cells. Results from this novel signal transduction microarray analysis revealed significant up- or downregulation of human cellular protein kinases, metalloproteases and one phosphatase. Radioactively labeled complex cDNA-probes from HCV Replicon cells Huh-9-13, Huh-5-15, and Huh-11-7 were hybridized to cDNA-arrays and compared to hybridizations with cDNA-probes from Huh-pcDNA control cells which did not contain HCV Replicons. Surprisingly it was found that the following cellular targets are significantly up- or downregulated:

| | |
|---|---|
| beta-adrenergic receptor kinase 1 (NM_001619): | 2.7-3.5 fold stronger |
| Mitogen activated protein kinase activated | 2.2-3.0 fold stronger |
| protein kinase 5 (AF032437): | |
| Insulin-stimulated protein kinase 1 (U08316): | 2.2-3.1 fold stronger |
| TRK E (NM_013994): | 3.2-10.3 fold stronger |
| Human p21-activated protein kinase 2 | 1.8-2.7 fold stronger |
| (U24153): | |
| PKC-mu (X75756): | 2.3-3.2 fold weaker |
| PKC-theta (L01087): | 2.6-3.3 fold weaker |
| AMP-activated protein kinase beta 2 subunit | 1.9-2.2 fold weaker |
| (AJ 224538): | |
| JNK2 (U09759): | 2.6-4.1 fold weaker |
| cdk4 (U37022) | 1.8-3.3 fold stronger |
| MEK5 (U25265) | 0.9-3.6 fold stronger |
| MKP-L (NM_007026): | 2.1-2.3 fold weaker |
| ADAM22 (NM_016351): | 2.5-3.8 fold weaker |
| ADAM17 (U92649): | 3.4-3.8 fold weaker |

Disclosed herein is the first report describing the role of human cellular proteins beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), ADAM17 (U92649) in the signal transduction of the HCV infection process. As a result of these discoveries, novel compounds and inhibitors against the above-mentioned human cellular protein kinases, metalloproteases and phosphatases may be found by the use of the inventive methods disclosed herein.

ADAM17 and ADAM22 are proteins of the ADAM family (proteins containing a disintegrin and metalloprotease domain). ADAM17 is also known as “Homo sapiens a disintegrin and metalloproteinase domain 17” and ADAM22 is know as “Homo sapiens metalloprotease-like, disintegrin-like, cysteine-rich protein 2 epsilon”.

As used herein, the term “inhibitor” refers to any compound capable of downregulating, decreasing, suppressing or otherwise regulating the amount and/or activity of the human cellular proteins beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649). Generally, human cellular protein inhibitors may be proteins, oligo- and polypeptides, nucleic acids, small chemical molecules, or other chemical moieties.

As used herein, the term “regulating expression and/or activity” generally refers to any process that functions to control or modulate the quantity or activity (functionality) of a cellular component. Static regulation maintains expression and/or activity at some given level. Upregulation refers to a relative increase in expression and/or activity. Accordingly downregulation refers to a relative decrease in expression and/or activity. In the present invention, regulation is preferably downregulation of a cellular component. As used herein, downregulation is synonymous with inhibition of a given cellular component's activity.

Therapeutics, pharmaceutically active agents or inhibitors, respectively, may be administered to cells from an individual in vitro, or may involve in vivo administration to the individual. The term “individual” preferably refers to mammals and most preferably to humans. Routes of administration of pharmaceutical preparations to an individual may include oral and parenteral, including dermal, intradermal, intragastral, intracutan, intravasal, intravenous, intramuscular, intraperitoneal, intranasal, intravaginal, intrabuccal, percutan, rectal, subcutaneous, sublingual, topical or transdermal application, but are not limited the these ways of administration. For instance, the preferred preparations are in administratable form which is suitable for oral application. These administratable forms, for example, include pills, tablets, film tablets, coated tablets, capsules, powders and deposits. Administration to an individual may be in a single dose or in repeated administrations, and may be in any of a variety of physiologically acceptable salt forms, and/or with an acceptable pharmaceutical carrier, binder, lubricant, excipient, diluent and/or adjuvant. Pharmaceutically acceptable salt forms and standard pharmaceutical formulation techniques are well known to persons skilled in the art.

As used herein, a “pharmaceutically effective amount” of a human cellular protein kinase, metalloprotease or phosphatase inhibitor is an amount effective to achieve the desired physiological result, either in cells treated in vitro or in a subject treated in vivo. Specifically, a pharmaceutically effective amount is an amount sufficient to inhibit, for some period of time, one or more of the clinically defined pathological processes associated with the viral infection. The effective amount may vary depending on the specific human cellular protein kinase, metalloprotease or phosphatase inhibitor selected, and is also dependent on a variety of factors and conditions related to the subject to be treated and the severity of the infection. For example, if the inhibitor is to be administered in vivo, factors such as the age, weight and health of the patient as well as dose response curves and toxicity data obtained in pre-clinical animal work would be among those considered. If the inhibitor is to be contacted with the cells in vitro, one would also design a variety of pre-clinical in vitro studies to assess such parameters as uptake, half-life, dose, toxicity, etc. The determination of a pharmaceutically effective amount for a given agent is well within the ability of those skilled in the art.

It is also apparent to a person skilled in the art that detection includes any method known in the art useful to indicate the presence, absence, or amount of a detection target. Such methods may include, but are not limited to, any molecular or cellular techniques, used singularly or in combination, including, but not limited to: hybridization and/or binding techniques, including blotting techniques and immunoassays; labeling techniques (chemiluminescent, calorimetric, fluorescent, radioisotopic); spectroscopic techniques; separations technology, including precipitations, electrophoresis, chromatography, centrifugation, ultrafiltration, cell sorting; and enzymatic manipulations (e.g., digestion).

The present disclosure teaches for the first time the up- or downregulation of a group of human cellular protein kinases, metalloproteases and a phosphatase specifically involved in the viral infection of Hepatitis C virus. Thus, the present invention is also directed to a method useful for detecting novel compounds useful for prophylaxis and/or treatment of HCV infections.

Methods of the present invention identify compounds useful for prophylaxis and/or treatment of HCV infections by screening a test compound, or a library of test compounds, for its ability to inhibit any one or more of the group of human cellular protein kinases, metalloproteases or phosphatases identified herein as characteristically up- or downregulated during HCV growth and RNA replication inside a cell. A variety of assay protocols and detection techniques are well known in the art and easily adapted for this purpose by a skilled practitioner. Such methods include, but are not limited to, high throughput assays (e.g., microarray technology, phage display technology), and in vitro and in vivo cellular and tissue assays.

In a related aspect, the present invention provides, in view of the discovery of human cellular protein kinases, metalloproteases and phosphatases specifically involved in the HCV infection process, an assay component specially useful for detecting HCV in an individual or in cells. Preferably the assay component comprises oligonucleotides immobilized on a solid support capable of detecting activity of one or more of the human cellular protein kinases, metalloproteases or phosphatase comprising:
the kinases beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1 (NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265);
the metalloproteases ADAM22 (NM_{—}016351), ADAM17 (U92649); and
the phosphatase MKP-L (NM_{—}007026).

Preferably the solid support would contain oligonucleotides of sufficient quality and quantity to detect all of the above-mentioned human cellular proteins (e.g., a nucleic acid microarray).

Similarly, it is an object of the present invention to provide an assay component specially useful for screening compounds useful for the prophylaxis and/or treatment of HCV infections. One preferred assay component comprises oligonucleotides that encode one or more human cellular protein kinases: beta-adrenergic receptor kinase (X61157), Mitogen activated protein kinase activated protein kinase (AF032437), Insulin-stimulated protein kinase 1 (U08316), TRK E (X74979), PKC-mu (X75756), PKC-theta (L01087), AMP-activated protein kinase beta 2 subunit (AJ 224538), JNK2 (L31951), Human p21-activated protein kinase, and PAK2 (U24153); or metalloproteases: ADAM22 (AF155382) and ADAM17 (XM_{—}002270); or the Phosphatase MKP-L, (NM_{—}007026) immobilized on a solid support.

In another embodiment, the assay component comprises peptide fragments of one or more of the above-identified human cellular proteins immobilized on a solid support. Once again the most preferred solid support embodiment would contain polymers of sufficient quality and quantity to detect all of the above-mentioned human cellular protein kinases, metalloproteases and the phosphatase (e.g., a nucleic acid or a peptide microarray). A variety of supports and constructions of the same for the methods disclosed herein are well known in the art and easily adapted for this purpose by a skilled practitioner (cf., for example: Marschall, 1999 “Do-it-yourself gene watching” Science 286, 444-447; Service 2000 “Protein arrays step out of DNA's shadow” Science 289, 1673).

It is preferred that mRNA is measured as an indication of expression. Methods for assaying for mRNA include, but are not limited to, Nothern blots, slot blots, dot blots, and hybridization to an ordered array of oligonucleotides. Nucleic acid probes useful for assay of a sample are preferably of sufficient length to specifically hybridize only to appropriate, complementary transcripts. Typically the oligonucleotide probes will be at least 10 to 25 nucleotides in length. In some cases longer probes of at least 30, 40, or 50 up to 2500 nucleotides will be desirable.

The polypeptide product of gene expression may be assayed to determine the amount of expression as well. Methods for assaying for a protein include, but are not limited to, western blot, immuno-precipitation, radioimmuno assay, and peptide immobilization in an ordered array. It is understood, however, that any method for specifically and quantitatively measuring a specific protein or mRNA product can be used.

A variety of supports upon which nucleic acids or peptides can be immobilized are known in the art, for example filters, or polyvinyl chloride dishes. Any solid surface to which oligonucleotides or peptides can be bound, either directly or indirectly, either covalently or non-covalently, can be used. A preferred solid support is a microarray membrane filter or a “biochip”. These contain particular polymer probes in predetermined locations on the array. Each predetermined location may contain more than one molecule of the probe, but each molecule within the predetermined location has an identical sequence.

The present invention incorporates by reference in their entirety techniques well known in the field of molecular biology. These techniques include, but are not limited to, techniques described in the following publications:
Ausubel, F. M. et al. eds., Short Protocols In Molecular Biology 4^{th} Ed. 1999, John Wiley & Sons, New York (ISBN 0-471-32938-X);
Old, R. W. & S. B. Primrose “Principles of Gene Manipulation: An Introduction To Genetic Engineering” 3^{rd} Ed. 1985, Blackwell Scientific Publications, Boston. Studies in Microbiology: V.2, 409 pp. (ISBN 0-632-01318-4);
Miller, J. H. & M. P. Calos eds., “Gene Transfer Vectors For Mammalian Cells” 1987, Cold Spring Harbor Laboratory Press, New York 169 pp. (ISBN 0-87969-198-0);
Mayer, R. J. & J. H. Walker eds. “Immunochemical Methods In Cell and Molecular Biology” 1987, Academic Press, London. 325 pp. (ISBN 0-12480-855-7);
Sambrook, J. et al. eds., “Molecular Cloning: A Laboratory Manual” 2^{nd} Ed. 1989, Cold Spring Harbor Laboratory Press, New York Vols. 1-3. (ISBN 0-87969-309-6); and
Winnacker, E. L. “From Genes To Clones: Introduction To Gene Technology” 1987 VCH Publishers, New York (translated by Horst Ibelgaufts) 634 pp. (ISBN 0-89573-614-4).

The present invention further incorporates by reference in their entirety techniques well known in the field of microarray construction and analysis. These techniques include, but are not limited to, techniques described in the following patents and patent applications describing array of biopolymeric compounds and methods for their fabrication:

U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,559,895; 5,624,711; 5,639,603; 5,658,734; 5,807,522; 6,087,102; WO 93/17126; WO 95/11995; WO 95/35505; EP 742 287; and EP 799 897.

Techniques also include, but are not limited to, techniques described in the following patents and patent application describing methods of using arrays in various applications:

U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,994,076; 6,033,860; 6,040,138; 6,040,140; WO 95/21265; WO 96/31622; WO 97/10365; WO 97/27317; EP 373 203; and EP 785 280.

It is readily apparent to those skilled in the art that other suitable modifications and adaptations of the compositions and methods of the invention described herein are evident and may be made without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLES

### Materials and Methods

1. Generation of cDNA-Arrays on Membranes

In order to manufacture cDNAs-arrays on membranes, the following strategy was pursued: cDNAs encoding parts of or full length proteins of interest—in the following referred to as “target cDNAs”—were cloned into the plasmid Bluescript II KS+(Stratagene, USA). Large scale purifications of these plasmids were performed according to standard techniques and 200 μl aliquots (1 μg/μl plasmid concentration) were transferred into appropriate 96 well plates. Plates were closed with sealing tape and chilled on ice for 5 minutes after incubation for 10 minutes at 95° C. 10 μl of 0.6N NaOH were added and the mix was stored for 20 minutes at room temperature before addition of 10 μl 2.5M Tris-HCl pH 7.1 and 20 μl 40×SSC. Target cDNAs were spotted onto Nylon or Nitrocellulose membranes using a BioGrid (BioRobotics, UK) equipped with a 0.7 mm pintool. In this way, between 200 ng and 350 ng of plasmids encoding target cDNAs were transferred onto the membranes and crosslinked to the membranes by ultraviolet light (1.2×10⁵ μJ/cm²). The arrays were stored for use in subsequent experiments at at 4° C.

2. Cellular HCV RNA Replication System

Huh-pcDNA3, Huh-9-13, Huh-5-15 and Huh-11-7 cells were grown in DMEM supplemented with 10% FCS, 2 mM Glutamine, Penicillin (100 IU/ml) / Streptomycin (100 μg/ml) and 1×nonessential amino acids in the presence of 1 mg/ml G418. Cells were routinely passaged three times a week at a dilution of 1:3 or 1:2.

3. Lysis of Cells, and Isolation of Total RNA

Huh-pcDNA3, Huh-9-13, Huh-5-15 and Huh-11-7 cells were seeded at 5×10⁵ cells per 10 cm plate in medium without G148. The medium was changed 3 days after plating and cells were harvested 5 days after plating by lyzing the cells directly on the plate with 4 ml of Tri reagent (Molecular Research Center, Inc., USA). The lysates were stored at room temperature for 5 minutes and then centrifuged at 12000×g for 15 minutes at 4° C. The supernatant was mixed with 0,1 ml of 1-Bromo-3-chloropropane per 1 ml of Tri reagent and vigorously shaken. The suspension was stored for 5 minutes at room temperature and then centrifuged at 12000×g for 15 minutes at 4° C. The colorless upper phase was transferred into new tubes, mixed with 5 μl of polyacryl-carrier (Molecular Research Center Inc., USA) and with 0.5 ml of isopropanol per 1 ml of Tri reagent and vigorously shaken. The samples were stored at room temperature for 5 minutes and then centrifuged at 12000×g for 8 minutes at 4° C. The supernatant was removed and the RNA pellet washed twice with 1 ml of 75% Ethanol. The pellet was dried and resuspended in 25 μl of RNase-free buffer per initial 1 ml lysate.

4. Preparation of Radioactively Labelled cDNA Probes from RNA

In order to obtain a radioactively labeled cDNA probe, RNA was transcribed into a cDNA-probe in the presence of radioactively labeled dATP. 12 μl double distilled DEPC treated H₂O containing 1 μg of primer TXN: 5′-TTT TTT TTT TTT TTT TVN-3′ (SEQ ID NO:30, wherein T is dTTP; N is dATP, dCTP, dGTP or dTTP; V is dATP, dCTP or dGTP) and total RNA (6 μg) were shaken between 5 and 15′ at 60° C. and then incubated at 4° C. for 2 to 10 minutes. After centrifugation (30 seconds, 10000×g) 7 μl of a mix consisting of 100 μCi dATP-P³³ (Amersham, UK) which were dried under vacuum previously and resuspended in 4 μl first strand buffer (Life Technologies, USA), 2 μl 0.1M DTT and 1 μl labeling solution (4 mM dCTP, dGTP, dTTP each and 80 μM dATP final concentration) were added. Following the addition of 1 μl Superscript II reverse transcriptase (Life Technologies, USA) the reaction was incubated for 10 minutes at room temperature and then for 60 minutes at 38° C. Subsequently, the reaction was vigorously shaken for 30 minutes at 68° C. after adding 5 μl 0.5M EDTA and 25 μl 0.6M NaOH.

Unincorporated nucleotides were removed from the labeling reaction using ProbeQuant G-50 columns (Amersham, UK). The column was vigorously shaken and centrifuged for 1 minute at 735×g in an appropriate reaction tube after bottom closure and lid were removed. The column was placed into a new reaction tube, the probe was applied onto the center of the column material and the column was centrifuged for 2 minutes at 735×g. The flow-trough was transferred into new reaction tubes and filled up to a volume of 100 p1 with 10 mM Tris, pH 7.4, 1 mM EDTA. The probe was precipitated by centrifugation for 15 minutes at 12000×g after 4 μl of 5M NaCl, 1 μl polyacryl-carrier (Molecular Research Center Inc., USA) and 250 μl Ethanol were added. The supernatant was discarded and the pellet dried before starting with the hybridisation.

5. Hybridisation of Radioactively Labeled cDNA-Probes to cDNA-Arrays

The pellet was resuspended in 10 μl C₀T DNA (1 μg/μl, Roche Diagnostics, Germany), 10 μl yeast tRNA (1 μg/μl Sigma, USA) and 10 μl polyA (1 μg/μl, Roche Diagnostics, Germany). Herring sperm DNA was added to a final concentration of 100 μg/ml and the volume was filled up to 100 μl with 5 μl 10% SDS (Sodiumdodecylsulfate), 25 μl 20×SSPE and double distilled H₂O. The mix was put on 95° C. for 5 minutes, centrifuged for 30 seconds at 10000×g and vigorously shaken for 60 minutes at 65° C. A 1 μl aliquot of the probe was used to measure the incorporation of radioactive dATP with a scintillation counter. Probes with at least a total of 20×10⁶ cpm were used. The arrays were prehybridised for at least 3 hours at 65° C. in hybridisation solution in a roller bottle oven. After prehybridisation the radioactively labeled probe was added into the hybridisation solution and hybridisation was continued for 20 hours. The probe was discarded and replaced with wash solution A (2×SSC). The arrays were washed twice in wash solution A at room temperature in the roller oven. Afterwards, wash solution A was replaced by wash solution B (2×SSC, 0.5% SDS) preheated to 65° C. and arrays were washed twice for 30 minutes at 65° C. Then, wash solution B was replaced by wash solution C (0.5×SSC, 0.5% SDS) preheated to 65° C. and arrays were washed twice for 30 minutes at 65° C. The moist arrays were wrapped in airtight bags and exposed for 8 to 72 hours on erased phosphoimager screens (Fujifilm, Japan).

6. Analysis of cDNA-Arrays

The exposed phosphoimager screens were scanned with a resolution of 100μ and 16 bits per pixel using a BAS-1800 (Fujifilm, Japan). Files were imported into the computer program ArrayVision (Imaging Research, Canada). Using the program's features, the hybridisation signals of each target cDNA were converted into numbers. The strength of the hybridisation signals reflected the quantity of RNA molecules present in the probe. Differentially expressed genes were selected according to the ratio of their signal strength after normalization to the overall intensity of the arrays.

7. Analysis of Expression Levels by Northern Blot Experiment

Huh-pcDNA3, Huh-9-13, Huh-5-15 and Huh-1 1-7 cells were seeded at 5×10⁵ cells per 10 cm plate in medium without G148. Cells were harvested after 3 days by lyzing the cells directly on the plate with 4 ml of Tri reagent (Molecular Research Centre, Inc., USA). The lysates were stored at room temperature for 5 minutes and then centrifuged at 12000×g for 15 minutes at 4° C. The supernatant was mixed with 0,1 ml of 1-bromo-3-chloropropane per 1 ml of Tri reagent and vigorously shaken. The suspension was stored for 5 minutes at room temperature and then centrifuged at 12000×g for 15 minutes at 4° C. The colourless upper phase was transferred into new tubes, mixed with 5 μl of poly-acryl-carrier (Molecular Research Centre, Inc., USA) and with 0.5 ml of isopropanol per 1 ml of Tri reagent and vigorously shaken. The samples were stored at room temperature for 5 minutes and then centrifuged at 12000×g for 8 minutes at 4° C. The supernatant was removed and the RNA pellet washed twice with 1 ml of 75% Ethanol. The pellet was dried and resuspended in 25 μl of RNase-free water per inital 1 ml lysate. 8 μg of total RNA per sample was loaded onto formaldehyde-containing agarose gels (Sambrook et al. Cloning manual, CSHL press, 1989) and transferred to HYbond NX membranes (Amersham) overnight in 20×SSC (3M NaCl, 300 mM C₆H₅Na₃O₇×2 H₂O, pH 7.0) by capillary transfer. RNA was immobilized to the filter using UV-crosslinking (120 mJ/cm² for 25 seconds). Filters were hybridized to oligonucleotide probes or random-primed probes specific for the genes in question. Quantitation of signals was performed with a Fuji phosphoimager.

8. Verification of De-Regulated Genes by Quantitative Real-Time PCR

Quantitative RT-PCR was used to verify hits resulting from DNA macroarray experiments by exploiting the 5′-exonulease of Taq DNA polymerases to cleave the 5′ fluorescent label of an oligonucleotide. Total RNA was extracted from cell lines (Qiagen RNeasy Mini Kit, QIAGEN, Hilden) and was reverse transcribed with Superscript II (Invitrogen, Karlsruhe) according to the manufacturer's protocol with 5 μg of RNA as a template and oligodT primers. Subsequently, the cDNA was analysed on a ABI PRISM 7000 Sequence Detection System (Applied Biosystems, Darmstadt) with the 5′exonuclease assay by using the TaqMan Universal PCR Master Mix (#4324018, Applied Biosystems, Darmstadt) and non-extendible oligonucleotides. Gene-specific Taq Man probes were labelled with the reporter dye FAM™ at the 5′-end and the quencher dye TAMRA™ at the 3′ end of the probe. GAPDH and 18SrRNA were used as reference genes with TaqMan probes that were labelled with VIC™ and TAMRA™ accordingly.

Experimental conditions were 2 minutes 50° C., 10 minutes 95° C., followed by 40 cycles with 15 seconds at 95° C. and 1 minute at 60° C. Primer Express software was used to design primers with a melting temperature of 58-60° C. amplifying an amplicon of a maximum length of 150 bp.

9. Analysis of Expression Levels by Western Blot Experiments

Huh-pcDNA3, Huh-9-13, Huh-5-15 and Huh-11-7 cells were seeded at 5×10⁵ cells per 10 cm plate in medium without G148. Cells were harvested after 3 days by the addition of 500 μl of 1×SDS sample buffer (62.5 mM Tris-HCl pH 6.8, 2% w/v SDS, 10% glycerol, 50 mM DTT, 0.01% bromphenol blue) or RIPA lysis buffer. Lysates were separated on SDS-poly acrylamide gels and proteins transfered to nitrocellulose. Western blotting was performed with the appropriate antibodies according to the manufacturers instructions.

11. Increased Expression of GRK2 (G Protein Coupled Receptor Kinase 2, Beta-Adrenergic Receptor Kinase 1, βARK1) mRNA in Subgenomic Replicon Lines was Confirmed by Quantitative Real-Time PCR.

Materials and Methods

Cellular HCV RNA Replication System.

Subgenomic HCV replicon lines Huh-9-13, Huh-5-15, Huh-1 1-7 (Science 1999, 285, 110-113), replicon line Huh-5-2 (carries the persistant replicon 13891uc-ubi-neo/NS3-3′/5.1 and expresses luciferase as a reporter (J. Virol. 2001, 75, 4614-24)) and the non-replicon control line Huh-pcDNA3 were cultured in Dulbecco's modified Eagle's medium supplemented with 1 mM sodium pyruvate, 2 mM glutamine, penicillin/streptomycin, 10% fetal bovine serum (DMEM/10% FBS) and 1×nonessential amino acids in the presence of 1 mg/ml G418. Cells were routinely passaged three times a week at a dilution of 1:3 or 1:2. Huh7 cells were grown in DMEM/10% FBS.

Confirmation of Array Results by Quantitative Real-Time PCR.

Quantitative RT-PCR was used to verify hits identified by DNA micro array experiments. Total RNA was extracted with Trizol Reagent (#15596-018, Invitrogen life technologies) and was reverse transcribed with Superscript II (Invitrogen) according to the manufacturer's protocol with 2 μg of RNA as a template and oligo-dT primers. Subsequently, the cDNA was analysed on a ABI PRISM 7000 Sequence Detection System (Applied Biosystems) using the TaqMan Universal PCR Master Mix (#4324018, Applied Biosystems). To measure the expression of GRK2, the TaqMan® Pre-Developed Assay Reagent (ID: Hs00176395_m1) was labelled with reporter dye FAM™ at the 5′-end and quencher dye TAMRA™ at the 3′ end of the probe. GAPDH was used as reference gene with TaqMan probes labelled with VIC™ and TAMRA™. Cycling conditions were 2 minutes at 50° C., 10 minutes at 95° C., followed by 40 cycles with 15 seconds at 95° C. and 1 minute at 60° C.

Results

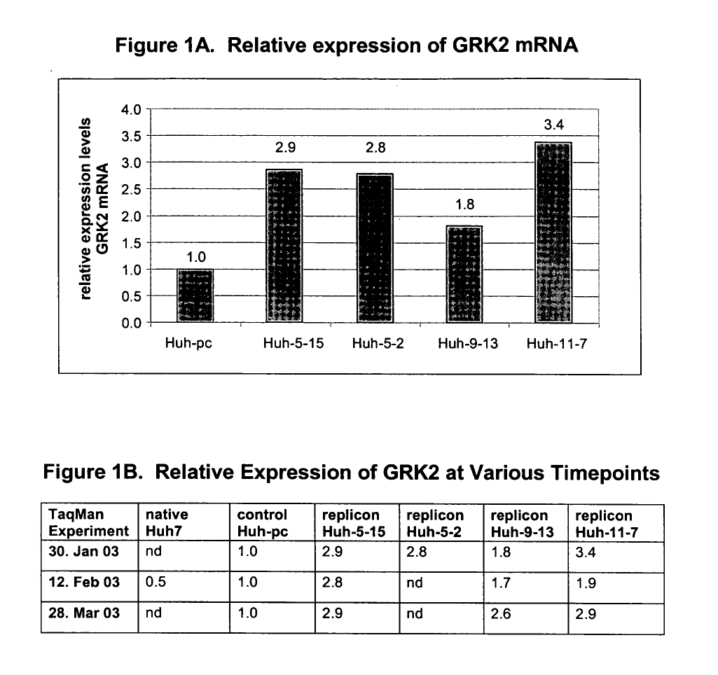

Total RNA was isolated from four different subconfluent subgenomic replicon lines or control lines in three independent experiments and GRK2-specific RNA expression levels were assessed by quantitative RT-PCR. A two- to three-fold higher expression of GRK2 RNA was observed in replicon lines (Huh-9-13, Huh-5-15, Huh-1 1-7, Huh-5-2) compared to the control cell line (Huh-pcDNA3) or native Huh7 cells (FIGS. 1A, 1B). This hints at a possible correlation between GRK2 function and HCV RNA replication or expression of HCV non-structural proteins.

12. Reduction of G Protein Coupled Receptor Kinase 2 (GRK2: Beta-Adrenergic Receptor Kinase 1, βARK1 ) Levels Correlates with a Reduction of HCV RNA Replication in a Subgenomic Replicon Cell Line.

Materials and Methods

Cellular HCV RNA Replication System.

Replicon line Huh-5-2 (carries the persistant replicon I389luc-ubi-neo/NS3-3′/5.1 and expresses luciferase as a reporter (J. Virol. 2001, 75, 4614-24)) and the non-replicon control line Huh-pcDNA3 were cultured in Dulbecco's modified Eagle's medium supplemented with 1 mM sodium pyruvate, 2 mM glutamine, penicillin/streptomycin, 10% fetal bovine serum (DMEM/10% FBS) and 1×nonessential amino acids in the presence of 1 mg/ml G418. Cells were routinely passaged three times a week at a dilution of 1:3 or 1:2.

Transient Transfection of Huh-5-2 Cells with siRNA Oligonucleotides.

Huh-5-2 replicon cells were seeded at 6×10⁴ cells per well in 6 well plates in DMEM/10% FBS without G418 and transfected using Oligofectamine (Invitrogen life technologies) with 50 nM of the following sequence specific siRNA oligoduplexes against GRK2 (siRNA-A: target sequence MG TAC GAG MG CTG GAG ACG (SEQ ID NO: 30); siRNA-C: target sequence MC ATC CTT CTG GAC GAG CAT (SEQ ID NO:31), against the luciferase gene encoded by the Huh-5-2 replicon (siRNA-Luc: target sequence MC GTA CGC GGA ATA CTT CGA (SEQ ID NO: 32)) or a control siRNA (GL3: target sequence MC TTA CGC TGA GTA CTT CGA (SEQ ID NO: 33))). Cells were lysed on day 3 after transfection (first transfection) or trypsinized and seeded on day 2 post transfection, transfected again the next day (second transfection) and harvested 3 days later. Cell viability was monitored on the day of lysis by counting or Alamar Blue assay. Cells were lysed in 250 μl of 1% Triton X100, 100 mM Tris-HCl pH 7.5. Luciferase activity in 50 μl aliquots of the lysates was determined with BrightGlo reagent (Promega) according to the manufacturer's protocol. For Western analysis, lysates were mixed with 3×SDS sample buffer (187.5 mM Tris-HCl pH 6.8, 6% SDS, 30% glycerol, 150 mM DTT, 0.03% bromphenol blue), resolved by 10% SDS-polyacrylamide gel electrophoresis and transferred to nitrocellulose membranes. GRK2 was visualized by immunoblotting with anti-GRK2 (Santa Cruz C-15), the appropriate secondary antibody and enhanced chemiluminescence reagent (Amersham). Blots were reprobed with anti-tubulin antibody (Sigma, clone 5-1-2) as a loading control.

Results

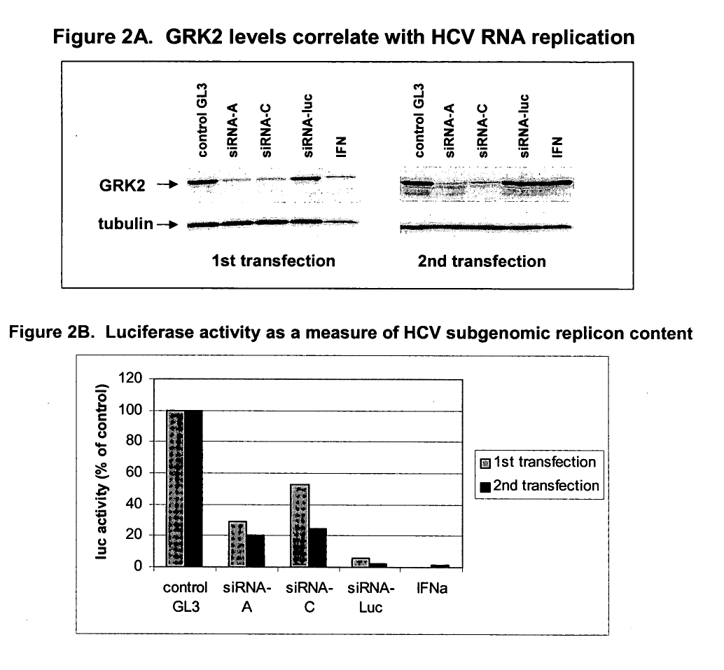

To validate the relevance of GRK2 upregulation for HCV replication, siRNA technology (Nature 2001 May 24;411 (6836):494-8) was used to decrease the levels of GRK2 in a replicon cell line. FIG. 2A demonstrates a significant reduction of GRK2 protein levels in Huh-5-2 cells transfected with GRK2 specific siRNA oligo-duplexes (FIG. 2A, siRNA-A, siRNA-C) compared to cells transfected with luciferase specific siRNA oligos (FIG. 2A siRNA-Luc) or a non-silencing control duplex RNA (FIG. 2A, control GL3). In parallel, replicon RNA content was determined by measuring luciferase activity in lysates of transfected Huh-5-2 cells. The 5-2 replicon line carries a bicistronic replicon that expresses a firefly luciferase—ubiquitin—neomycin phosphotransferase fusion protein under the control of the HCV 5′ UTR, in addition to the HCV nonstructural proteins under the control of the EMCV IRES element (J. Virol. 75:4614-4624, 2001). The amount of autonomously replicating HCV RNA is therefore directly related to the levels of luciferase activity. Transfection with GRK2-specific siRNAs led to a significant reduction of luciferase activity compared to transfection with control GL3 siRNA, especially after two subsequent transfections (FIG. 2B, 2^{nd} transfection, siRNA-A, siRNA-C vs. control GL3). Transfections with siRNA targeting the luciferase sequence of the replicon RNA (FIG. 2B, siRNA-Luc) and treatment of the cells with 100 units/ml of interferon alpha (IFNa) served as positive controls. In summary, reduction of GRK2 levels appeared to correlate with diminished HCV RNA replication, suggesting a possible requirement of GRK2 for HCV replication.

1. A method for detecting compounds useful for the prophylaxis and/or treatment of Hepatitis C virus infections, the method comprising the following steps:
a) contacting a test compound with at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1 (NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), ADAM17 (U92649); and
b) determining the activity of said human cellular protein kinase, metalloprotease or phosphatase.

2. A method for detecting Hepatitis C virus infections in an individual, the method comprising the following steps:
a) providing a sample from said individual; and
b) determining the activity in said sample of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1 (NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

3. A method for detecting Hepatitis C virus infections in cells, the method comprising the following steps:
a) providing said cells; and
b) determining the activity in said cells of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

4. An antibody that binds to a human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

5. The antibody of claim 4, wherein said antibody is a monoclonal or polyclonal antibody.

6. A method for treating Hepatitis C virus infection in an individual, the method comprising the step of administering a pharmaceutically effective amount of an agent which inhibits at least partially the activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

7. A method for regulating the production of Hepatitis C virus in cells, the method comprising the step of administering a pharmaceutically effective amount of an agent to said cells wherein said agent inhibits at least partially the activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1 (NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

8. The method according to claim 6 or 7, wherein the agent is an antibody which binds to at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

9. The method according to claim 8, wherein said antibody is a monoclonal or polyclonal antibody.

10. A solid support useful for detecting Hepatitis C virus infections in an individual, the solid support comprising an immobilized oligonucleotide, wherein said oligonucleotide is capable of detecting activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

11. A solid support useful for detecting Hepatitis C virus infections in cells, the solid support comprising an immobilized oligonucleotide, wherein said oligonucleotide is capable of detecting activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

12. A solid support useful for screening compounds useful for the prophylaxis and/or treatment of Hepatitis C virus infections in an individual, the solid support comprising at least one immobilized oligonucleotide, wherein said oligonucleotide encodes one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1(NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

13. A solid support useful for screening compounds useful for the prophylaxis and/or treatment of Hepatitis C virus infections in an individual, the solid support comprising at least one immobilized human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1 (NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).

14. A composition useful for the prophylaxis and/or treatment of an individual afflicted with Hepatitis C virus, the composition comprising at least one agent capable of inhibiting activity of at least one human cellular protein kinase, metalloprotease or phosphatase selected from the group consisting of beta-adrenergic receptor kinase 1 (NM_{—}001619), Mitogen activated protein kinase activated protein kinase 5 (AF032437), Insulin-stimulated protein kinase 1 (U08316), Discoidin domain receptor family, member 1 (NM_{—}013994), Protein Kinase C, mu (X75756), Protein Kinase C, theta (L01087), AMP-activated protein kinase beta 2 subunit (AJ 224538), JNK2 (U09759), Human p21-activated protein kinase 2 (U24153), cyclin-dependent kinase 4 (U37022), MEK5 (U25265), MKP-L (NM_{—}007026), ADAM22 (NM_{—}016351), and ADAM17 (U92649).


SEQUENCE LISTING (33 sequences)

SEQ ID NO: 1 - protein, 471 aa, Homo sapiens [sequence omitted]

SEQ ID NO: 2 - DNA, 1968 bp, Homo sapiens
ccagcctagagccgcccgccgaagcagagccggcgccggggtcctcatccccaccggtcccgaggggcggctgctgcccgtcgccacgaggcccaggggcccgagtgccgagccctttgctccctcggccgcgcggggacagggctgctgagcagcctccgcctctcccggctgtgggggccccactgagtatgtcggaggagagcgacatggacaaagccatcaaggaaacttccattttagaagaatacagtatcaattggactcagaagctgggagctggaattagtggtccagttagagtctgtgtaaagaaatctactcaagaacggtttgcgctgaaaattcttcttgatcgtccaaaagctagaaatgaggtacgtctgcacatgatgtgtgccacacacccaaacatagttcagattattgaagtgtttgctaacagtgtccagtttccccatgagtccagccctagggcccgactcttaattgtaatggagatgatggaagggggagagctatttcacagaatcagccagcaccggcactttacagagaagcaagccagccaagtaacaaagcagatagctttggctctgcggcactgtcacttgttaaacattgcgcacagagacctcaagcctgaaaatctgctttttaaggataactctttggatgccccagtgaagttgtgtgactttggatttgccaagattgaccaaggtgacttgatgacaccccagttcaccccttattatgtagcaccccaggtactggaggcgcaaagaaggcatcagaaggagaaatctggcatcatacctacctcaccgacgccctacacttacaacaagagctgtgacttgtggtccctaggggtgattatctatgtgatgctgtgcggataccctcctttttactccaaacaccacagccggactatcccaaaggatatgcgaagaaagatcatgacaggcagttttgagttcccagaggaagagtggagtcagatctcagagatggccaaagatgttgtgaggaagctcctgaaggtcaaaccggagaggagactcaccatcgagggagtgctggaccacccctggctcaattccaccgaggccctggataatgtgctgccttctgctcagctgatgatggacaaggcagtggttgcaggaatccagcaggctcacgcggaacagttggccaacatgagaatccaggatctgaaagtcagcctcaaacccctgcactcagtgaacaaccccattctgcggaagaggaagttacttggcaccaagccaaaggacagtgtctatatccacgaccatgagaatggagccgaggattccaatgttgccttggaaaaactccgagatgtgattgctcagtgtattctcccccaggctggagagaatgaagatgagaaactgaatgaagtaatgcaggaggcttggaagtataaccgggaatgcaaactcctaagagatactctgcagagcttcagctggaatggtcgtggattcacagataaagtagatcgactaaaactggcagaaattgtgaagcaggtgatagaagagcaaaccacgtcccacgaatcccaataatgacagcttcagactttgtttttttaacaatttgaaaaattattctttaatgtataaagtaattttatgtaaattaataaatcataatttcatttccacattgattaaagctgctgtatagatttagggtgcaggacttaataatagtatagttattgtttgtttttaagaaaagctcagttctagagacatactattactttaggactgtgtagttgtatatttgtaagatgacagatgatgctgtcaagcaatattgttttatttgtaataaaatatacaaaaatcacttgccagcagtagaaaaaggaccgactataccgacctttctgattagtaaacagttgaatcaaggactctg

SEQ ID NO: 3 - protein, 740 aa, Homo sapiens
MPLAQLADPWQKMAVESPSDSAENGQQIMDEPMGEEEINPQTEEVSIKEIAITHHVKEGHEKADPSQFELLKVLGQGSFGKVFLVKKISGSDARQLYAMKVLKKATLKVRDRVRTKMERDILVEVNHPFIVKLHYAFQTEGKLYLILDFLRGGDLFTRLSKEVMFTEEDVKFYLAELALALDHLHSLGIIYRDLKPENILLDEEGHIKLTDFGLSKESIDHEKKAYSFCGTVEYMAPEVVNRRGHTQSADWWSFGVLMFEMLTGTLPFQGKDRKETMTMILKAKLGMPQFLSPEAQSLLRMLFKRNPANRLGAGPDGVEEIKRHSFFSTIDWNKLYRREIHPPFKPATGRPEDTFYFDPEFTAKTPKDSPGIPPSANAHQLFRGFSFVAITSDDESQAMQTVGVHSIVQQLHRNSIQFTDGYEVKEDIGVGSYSVCKRCIHKATNMEFAVKIIDKSKRDPTEEIEILLRYGQHPNIITLKDVYDDGKYVYVVTELMKGGELLDKILRQKFFSEREASAVLFTITKTVEYLHAQGVVHRDLKPSNILYVDESGNPESIRICDFGFAKQLRAENGLLMTPCYTANFVAPEVLKRQGYDAACDIWSLGVLLYTMLTGYTPFANGPDDTPEEILARIGSGKFSLSGGYWNSVSDTAKDLVSKMLHVDPHQRLTAALVLRHPWIVHWDQLPQYQLNRQDAPHLVKGAMAATYSALNRNQSPVLEPVGRSTLAQRRGIKKITSTAL

SEQ ID NO: 4 - DNA, 2260 bp, Homo sapiens
atgccgctggcgcagctggcggacccgtggcagaagatggctgtggagagcccgtccgacagcgctgagaatggacagcaaattatggatgaacctatgggagaggaggagattaacccacaaactgaagaagtcagtatcaaagaaattgcaatcacacatcatgtaaaggaaggacatgaaaaggcagatccttcccagtttgaacttttaaaagtattagggcagggatcatttggaaaggttttcttagttaaaaaaatctcaggctctgatgctaggcagctttatgccatgaaggtattgaagaaggccacactgaaagttcgagaccgagttcggacaaaaatggaacgtgatatcttggtagaggttaatcatccttttattgtcaagttgcattatgcttttcaaactgaagggaagttgtatcttattttggattttctcaggggaggagatttgtttacacgcttatccaaagaggtgatgttcacagaagaagatgtcaaattctacttggctgaacttgcacttgctttagaccatctacatagcctgggaataatttatagagacttaaaaccagaaaatatacttcttgatgaagaaggtcacatcaagttaacagatttcggcctaagtaaagagtctattgaccatgaaaagaaggcatattctttttgtggaactgtggagtatatggctccagaagtagttaatcgtcgaggtcatactcagagtgctgactggtggtcttttggtgtgttaatgtttgaaatgcttactggtacactccctttccaaggaaaagatcgaaaagaaacaatgactatgattcttaaagccaaacttggaatgccacagtttttgagtcctgaagcgcagagtcttttacgaatgcttttcaagcgaaatcctgcaaacagattaggtgcaggaccagatggagttgaagaaattaaaagacattcatttttctcaacgatagactggaataaactgtatagaagagaaattcatccgccatttaaacctgcaacgggcaggcctgaagatacattctattttgatcctgagtttactgcaaaaactcccaaagattcacctggcattccacctagtgctaatgcacatcagctttttcgggggtttagttttgttgctattacctcagatgatgaaagccaagctatgcagacagttggtgtacattcaattgttcagcagttacacaggaacagtattcagtttactgatggatatgaagtaaaagaagatattggagttggctcctactctgtttgcaagagatgtatacataaagctacaaacatggagtttgcagtgaagattattgataaaagcaagagagacccaacagaagaaattgaaattcttcttcgttatggacagcatccaaacattatcactctaaaggatgtatatgatgatggaaagtatgtgtatgtagtaacagaacttatgaaaggaggtgaattgctggataaaattcttagacaaaaatttttctctgaacgagaggccagtgctgtcctgttcactataactaaaaccgttgaatatcttcacgcacaaggggtggttcatcgagacttgaaacctagcaacattctttatgtggatgaatctggtaatccggaatctattcgaatttgtgattttggctttgcaaaacagctgagagcggaaaatggtcttctcatgactccttgttacactgcaaattttgttgcaccagaggttttaaaaagacaaggctatgatgctgcttgtgatatatggagtcttggtgtcctactctatacaatgcttaccggttacactccatttgcaaatggtcctgatgatacaccagaggaaatattggcacgaataggtagcggaaaattctcactcagtggtggttactggaattctgtttcagacacagcaaaggacctggtgtcaaagatgcttcatgtagaccctcatcagagactgactgctgctcttgtgctcagacatccttggatcgtccactgggaccaactgccacaataccaactaaacagacaggatgcaccacatctagtaaagggtgccatggcagctacatattctgctttgaaccgtaatcagtcaccagttttggaaccagtaggccgctctactcttgctcagcggagaggtattaaaaaaatcacctcaacagccctgtgaagtgacctcagtgagatatttggatccatggtgtaaa

SEQ ID NO: 5 - protein, 912 aa, Homo sapiens
MSAPPVLRPPSPLLPVAAAAAAAAAALVPGSGPGPAPFLAPVAAPVGGISFHLQIGLSREPVLLLQDSSGDYSLAHVREMACSIVDQKFPECGFYGMYDKILLFRHDPTSENILQLVKAASDIQEGDLIEVVLSRSATFEDFQIRPHALFVHSYRAPAFCDHCGEMLWGLVRQGLKCEGCGLNYHKRCAFKIPNNCSGVRRRRLSNVSLTGVSTIRTSSAELSTSAPDEPLLQKSPSESFIGREKRSNSQSYIGRPIHLDKILMSKVKVPHTFVIHSYTRPTVCQYCKKLLKGLFRQGLQCKDCRFNCHKRCAPKVPNNCLGEVTINGDLLSPGAESDVVMEEGSDDNDSERNSGLMDDMEEAMVQDAEMAMAECQNDSGEMQDPDPDHEDANRTISPSTSNNIPLMRVVQSVKHTKRKSSTVMKEGWMVHYTSKDTLRKRHYWRLDSKCITLFQNDTGSRYYKEIPLSEILSLEPVKTSALIPNGANPHCFEITTANVVYYVGENVVNPSSPSPNNSVLTSGVGADVARMWEIAIQHALMPVIPKGSSVGTGTNLHRDISVSISVSNCQIQENVDISTVYQIFPDEVLGSGQFGIVYGGKHRKTGRDVAIKIIDKLRFPTKQESQLRNEVAILQNLHHPGVVNLECMFETPERVFVVMEKLHGDMLEMILSSEKGRLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWLQDYQTWLDLRELECKIGERYITHESDDLRWEKYAGEQRLQYPTHLINPSASHSDTPETEETEMKALGERVSIL

SEQ ID NO: 6 - DNA, 3742 bp, Homo sapiens
gaattccttctctcctcctcctcgcccttctcctcgccctcctcctcctcctcgccctcccctcccgatcctcatccccttgccctcccccagcccagggacttttccggaaagtttttattttccgtctgggctctcggagaaagaagctcctggctcagcggctgcaaaactttcctgctgccgcgccgccagcccccgccctccgctgcccggccctgcgccccgccgagcgatgagcgcccctccggtcctgcggccgcccagtccgctgctgcccgtggcggcggcagctgccgcagcggccgccgcactggtcccagggtccgggcccgggcccgcgccgttcttggctcctgtcgcggccccggtcgggggcatctcgttccatctgcagatcggcctgagccgtgagccggtgctgctgctgcaggactcgtccggggactacagcctggcgcacgtccgcgagatggcttgctccattgtcgaccagaagttccctgaatgtggtttctacggaatgtatgataagatcctgctttttcgccatgaccctacctctgaaaacatccttcagctggtgaaagcggccagtgatatccaggaaggcgatcttattgaagtggtcttgtcacgttccgccacctttgaagactttcagattcgtccccacgctctctttgttcattcatacagagctccagctttctgtgatcactgtggagaaatgctgtgggggctggtacgtcaaggtcttaaatgtgaagggtgtggtctgaattaccataagagatgtgcatttaaaatacccaacaattgcagcggtgtgaggcggagaaggctctcaaacgtttccctcactggggtcagcaccatccgcacatcatctgctgaactctctacaagtgcccctgatgagccccttctgcaaaaatcaccatcagagtcgtttattggtcgagagaagaggtcaaattctcaatcatacattggacgaccaattcaccttgacaagattttgatgtctaaagttaaagtgccgcacacatttgtcatccactcctacacccggcccacagtgtgccagtactgcaagaagcttctgaaggggcttttcaggcagggcttgcagtgcaaagattgcagattcaactgccataaacgttgtgcaccgaaagtaccaaacaactgccttggcgaagtgaccattaatggagatttgcttagccctggggcagagtctgatgtggtcatggaagaagggagtgatgacaatgatagtgaaaggaacagtgggctcatggatgatatggaagaagcaatggtccaagatgcagagatggcaatggcagagtgccagaacgacagtggcgagatgcaagatccagacccagaccacgaggacgccaacagaaccatcagtccatcaacaagcaacaatatcccactcatgagggtagtgcagtctgtcaaacacacgaagaggaaaagcagcacagtcatgaaagaaggatggatggtccactacaccagcaaggacacgctgcggaaacggcactattggagattggatagcaaatgtattaccctctttcagaatgacacaggaagcaggtactacaaggaaattcctttatctgaaattttgtctctggaaccagtaaaaacttcagctttaattcctaatggggccaatcctcattgtttcgaaatcactacggcaaatgtagtgtattatgtgggagaaaatgtggtcaatccttccagcccatcaccaaataacagtgttctcaccagtggcgttggtgcagatgtggccaggatgtgggagatagccatccagcatgcccttatgcccgtcattcccaagggctcctccgtgggtacaggaaccaacttgcacagagatatctctgtgagtatttcagtatcaaattgccagattcaagaaaatgtggacatcagcacagtatatcagatttttcctgatgaagtactgggttctggacagtttggaattgtttatggaggaaaacatcgtaaaacaggaagagatgtagctattaaaatcattgacaaattacgatttccaacaaaacaagaaagccagcttcgtaatgaggttgcaattctacagaaccttcatcaccctggtgttgtaaatttggagtgtatgtttgagacgcctgaaagagtgtttgttgttatggaaaaactccatggagacatgctggaaatgatcttgtcaagtgaaaagggcaggttgccagagcacataacgaagtttttaattactcagatactcgtggctttgcggcaccttcattttaaaaatatcgttcactgtgacctcaaaccagaaaatgtgttgctagcctcagctgatccttttcctcaggtgaaactttgtgattttggttttgcccggatcattggagagaagtctttccggaggtcagtggtgggtacccccgcttacctggctcctgaggtcctaaggaacaagggctacaatcgctctctagacatgtggtctgttggggtcatcatctatgtaagcctaagcggcacattcccatttaatgaagatgaagacatacacgaccaaattcagaatgcagctttcatgtatccaccaaatccctggaaggaaatatctcatgaagccattgatcttatcaacaatttgctgcaagtaaaaatgagaaagcgctacagtgtggataagaccttgagccacccttggctacaggactatcagacctggttagatttgcgagagctggaatgcaaaatcggggagcgctacatcacccatgaaagtgatgacctgaggtgggagaagtatgcaggcgagcagcggctgcagtaccccacacacctgatcaatccaagtgctagccacagtgacactcctgagactgaagaaacagaaatgaaagccctcggtgagcgtgtcagcatcctctgagttccatctcctataatctgtcaaaacactgtggaactaataaatacatacggtcaggtttaacatttgccttgcagaactgccattattttctgtcagatgagaacaaagctgttaaactgttagcactgttgatgtatctgagttgccaagacaaatcaacagaagcatttgtattttgtgtgaccaactgtgttgtattaacaaaagttccctgaaacacgaaacttgttattgtgaatgattcatgttatatttaatgcattaaacctgtctccactgtgcctttgcaaatcagtgtttttcttactggagcttcattttggtaagagacagaatgtatctgtgaagtagttctgtttggtgtgtcccattggtgttgtcattgtaaacaaactcttgaagagtcgattatttccagtgttctatgaacaactccaaaacccatgtgggaaaaaaatgaatgaggagggtagggaataaaatcctaagacacaaatgcatgaacaagttttaatgtatagttttgaatcctttgcctgcctggtgtgcctcagtatatttaaactcaagacaatgcacctagctgtgcaagacctagtgctcttaagcctaaatgccttagaaatgtaaactgccatatataacagatacatttccctctttcttataatactctgttgtactatggaaaatcagctgctcagcaacctttcacctttgtgtatttttcaataataaaaaatattcttgtcaaaaaaaaaaaaa

SEQ ID NO: 7 - protein, 706 aa, Homo sapiens [sequence omitted]

SEQ ID NO: 8 - DNA, 2754 bp, Homo sapiens
gaattccgccagccccgccagtccccgcgcagtccccgcgcagtcccagcgccaccgggcagcagcggcgccgtgctcgctccagggcgcaaccatgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggctgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacatttcagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaacccccggatggagcggctgatatcctgaatcttgcccctccagagacaggaaagaatttgccttgtccctgggaactggttcaagagacactgcttgggttcctttttcaacttggaaaaagaaagaaacactcaacaataaagactgagacccgttcgcccccatgtgacttttatctgtagcagaaaccaagtctacttcactaatgacgatgccgtgtgtctcgtctcctgacatgtctcacagacgctcctgaagttaggtcattactaaccatagttatttacttgaaagatgggtctccgcacttggaaaggtttcaagacttgatactgcaataaattatggctcttcacctgggcgccaactgctgatcaacgaaatgcttgttgaatcaggggcaaacggagtacagacgtctcaagactgaaacggccccattgcctggtctagtagcggatctcactcagccgcagacaagtaatcactaacccgttttattctattcctatctgtggatgggtaaatgctgggggccagccctggataggtttttatgggaattc

SEQ ID NO: 9 - protein, 272 aa, Homo sapiens
MGNTTSDRVSGERHGAKAARSEGAGGHAPGKEHKIMVGSTDDPSVFSLPDSKLPGDKEFVSWQQDLEDSVKPTQQARPTVIRWSEGGKEVFISGSFNNWSTKIPLIKSHNDFVAILDLPEGEHQYKFFVDGQWVHDPSEPVVTSQLGTINNLIHVKKSDFEVFDALKLDSMESSETSCRDLSSSPPGPYGQEMYAFRSEERFKSPPILPPHLLQVILNKDTNISCDPALLPEPNHVMLNHLYALSIKDSVMVLSATHRYKKKYVTTLLYKPI

SEQ ID NO: 10 - DNA, 819 bp, Homo sapiens
atgggaaacaccaccagcgaccgggtgtccggggagcgccacggcgccaaggctgcacgctccgagggcgcaggcggccatgccccggggaaggagcacaagatcatggtggggagtacggacgaccccagcgtgttcagcctccctgactccaagctccctggggacaaagagtttgtatcatggcagcaggatttggaggactccgtaaagcccacacagcaggcccggcccactgttatccgctggtctgaaggaggcaaggaggtcttcatctctgggtctttcaacaattggagcaccaagattccactgattaagagccataatgactttgttgccatcctggacctccctgagggagagcaccaatacaagttctttgtggatggacagtgggttcatgatccatcagagcctgtggttaccagtcagcttggcacaattaacaatttgatccatgtcaagaaatctgattttgaggtgttcgatgctttaaagttagattctatggaaagttctgagacatcttgtagagacctttccagctcacccccagggccttatggtcaagaaatgtatgcgtttcgatctgaggaaagattcaaatccccacccatccttcctcctcatctacttcaagttattcttaacaaagacactaatatttcttgtgacccagccttactccctgagcccaaccatgttatgctgaaccatctctatgcattgtccattaaggacagtgtgatggtccttagcgcaacccatcgctacaagaagaagtatgttactactctgctatacaagcccatttga

SEQ ID NO: 11 - protein, 424 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDHIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPPDAAVSSNATPSQSSSINDISSMSTEQTLASDTDSSLDASTGPLEGCR

SEQ ID NO: 12 - DNA, 1873 bp, Homo sapiens
caaactacgtgctgtacagctgcatcagctgctcgtagacatgtccagcagctggtcgaggtccacgccgcggtaggtgaagttgcggaaggtccggcgagggatctgaaacttgccccttacccttcgggatattgcaggacgctgcatcatgagcgacagtaaatgtgacagtcagttttatagtgtccaagtggcagactcaaccttcactgtcctaaaacgttaccagcagctgaaaccaattggctctggggcccaagggattgtttgtgctgcatttgatacagttcttgggataaatgttgcagtcaagaaactaagccgtccttttcagaaccaaactcatgcaaagagagcttatcgtgaacttgtcctcttaaaatgtgtcaatcataaaaatataattagtttgttaaatgtgtttacaccacaaaaaactctagaagaatttcaagatgtgtatttggttatggaattaatggatgctaacttatgtcaggttattcacatggagctggatcatgaaagaatgtcctaccttctttaccagatgctttgtggtattaaacatctgcattcagctggtataattcatagagatttgaagcctagcaacattgttgtgaaatcagactgcaccctgaagatccttgactttggcctggcccggacagcgtgcactaacttcatgatgaccccttacgtggtgacacggtactaccgggcgcccgaagtcatcctgggtatgggctacaaagagaacgttgatatctggtcagtgggttgcatcatgggagagctggtgaaaggttgtgtgatattccaaggcactgaccatattgatcagtggaataaagttattgagcagctgggaacaccatcagcagagttcatgaagaaacttcagccaactgtgaggaattatgtcgaaaacagaccaaagtatcctggaatcaaatttgaagaactctttccagattggatattcccatcagaatctgagcgagacaaaataaaaacaagtcaagccagagatctgttatcaaaaatgttagtgattgatcctgacaagcggatctctgtagacgaagctctgcgtcacccatacatcactgtttggtatgaccccgccgaagcagaagccccaccacctcaaatttatgatgcccagttggaagaaagagaacatgcaattgaagaatggaaagagctaatttacaaagaagtcatggattgggaagaaagaagcaagaatggtgttgtaaaagatcagcctccagatgcagcagtaagtagcaacgccactccttctcagtcttcatcgatcaatgacatttcatccatgtccactgagcagacgctggcctcagacacagacagcagtcttgatgcctcgacgggaccccttgaaggctgtcgatgataggttagaaatagcaaacctgtcagcattgaaggaactctcacctccgtgggcctgaaatgcttgggagttgatggaaccaaatagaaaaactccatgttctgcatgtaagaaacacaatgccttgccctactcagacctgataggattgcctgcttagatgataaaatgaggcagaatatgtctgaagaaaaaaattgcaagccacacttctagagattttgttcaagatcatttcagttgagcagttagagtaggtgaatttgtcaaattgtactagtgacagtttctcatcatctgtaactgttgagatgattgtgcatgtgaccacaaatgcttgcttggacttgcccatctagcactttggaaatcagtatttaaatgccaaataatcttccaggtagtgctgcttctgaagttatctcttaatcctcttaagtaatttgg

SEQ ID NO: 13 - protein, 525 aa, Homo sapiens [sequence omitted]

SEQ ID NO: 14 - DNA, 2019 bp, Homo sapiens
gaccttggcttgcccggggccatttcataattctgaatcatgtctgataacggagaactggaagataagcctccagcacctcctgtgcgaatgagcagcaccatctttagcactggaggcaaagaccctttgtcagccaatcacagtttgaaacctttgccctctgttccagaagagaaaaagcccaggcataaaatcatctccatattctcaggcacagagaaaggaagtaaaaagaaagaaaaggaacggccagaaatttctcctccatctgattttgagcacaccatccatgttggctttgatgctgttactggagaattcactggcatgccagaacagtgggctcgattactacagacctccaatatcaccaaactagagcaaaagaagaatcctcaggctgtgctggatgtcctaaagttctacgactccaacacagtgaagcagaaatatctgagctttactcctcctgagaaagatggccttccttctggaacgccagcactgaatgccaagggaacagaagcacccgcagtagtgacagaggaggaggatgatgatgaagagactgctcctcccgttattgccccgcgaccggatcatacgaaatcaatttacacacggtctgtaattgaccctgttcctgcaccagttggtgattcacatgttgatggtgctgccaagtctttagacaaacagaaaaagaagcctaagatgacagatgaagagattatggagaaattaagaactatcgtgagcataggtgaccctaagaaaaaatatacaagatatgaaaaaattggacaaggggcttctggtacagttttcactgctactgacgttgcactgggacaggaggttgctatcaaacaaattaatttacagaaacagccaaagaaggaactgatcattaacgagattctggtgatgaaagaattgaaaaatcccaacatcgttaactttttggacagttacctggtaggagatgaattgtttgtggtcatggaataccttgctggggggtcactcactgatgtggtaacagaaacagcttgcatggatgaagcacagattgctgctgtatgcagagagtgtttacaggcattggagtttttacatgctaatcaagtgatccacagagacatcaaaagtgacaatgtacttttgggaatggaaggatctgttaagctcactgactttggtttctgtgcccagatcacccctgagcagagcaaacgcagtaccatggtcggaacgccatactggatggcaccagaggtggttacacggaaagcttatggccctaaagtcgacatatggtctctgggtatcatggctattgagatggtagaaggagagcctccatacctcaatgaaaatcccttgagggccttgtacctaatagcaactaatggaaccccagaacttcagaatccagagaaactttccccaatatttcgggatttcttaaatcgatgtttggaaatggatgtggaaaaaaggggttcagccaaagaattattacagcatcctttcctgaaactggccaaaccgttatctagcttgacaccactgatcatggcagctaaagaagcaatgaagagtaaccgttaacatcactgctgtgggctcatactcttttttccattttctacaagaagccttttagtatatgaaaatgatgactctgttgggggtttaaagaaatggtctgcataacctgaatgaaagaaggaaatgactattctctgaagacaaccaagagaaaattggaaaagacaaggtatgactttgttatgaacccctgcttttaggggtccaggaagggatttgtgggacttgaattcactaggcttaggtctttcaggaaacaggctatcaggggcatttatcatgtgtgagattggattctacttgggtgatttggtggatagacccatgaatggcccctgggggttttcaatcttggattggaggtgggggtttcagagtgttgccacgtctagctcctctccc

SEQ ID NO: 15 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 16 - DNA, 4233 bp, Homo sapiens
  misc_feature (2320)..(2321): n is a, c, g, or t
ccctcctcccagtcgaagcacctcctgtccgcccctcagcgcatgggtggcggtcacgtgcccagaacgtccggcgttcgccccgccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgaggtaagtgcagtcccttcccaggaatgagaaccagtgcccgcccccctcacagctttccacgcgttcgtttcgcgagctggttatggaagggtcgctcaagggcgggaagtggggcctttgtggtcatgggaaagtataattttagggactgaggtgtaggatcttcgatgcaaggcatgtgtcatgtgtgatctttgtgcggggcgcgattgtcccaaaggaaaaagcgttttctattgcagggcctcacgtggctggaggggttggtattgagtcattgtgttatctctggggccggccccaaggaagactgggagcgggggatgggatgctggtggtgttctttgcgctttttttttgggagtccctttgttgctgcaggtcataccatcctaactctgtaagcgacttttggtgataggagtctgtgattgtagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggtgagaaggtggtggagggttgggcgtggggagtaaagggaaaagacagcctataggtggggtgtgatgatctgtagagaagtggggaccctgaggaaataatgagaggccatgttgggttaaaggggattgaaaagtgagcatttactctggtcaggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggtgagtggggttggtaggcattgagaggtggattgggacctttgtagtagaaccttctgggatttcaggtatggtgcctagtttccagtgcatctgtacctccccctttgaaactaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggtcagtagaaagatggtaccaaaatgggttctggttgggaataggagagtgattgcccgtagcaattgagaagtcatgtgcttcatgtgttcagtcaagcaagttgtgtttcatggtaacccatggggtccccatccattcttcctattccctttaggttgttacactctggtaccgagctcccgaagttcttctgcagtccacatatgcaacacctgtggacatgtggagtgttggctgtatctttgcagagatgtttcgtcgaaagtatgggacccacataccctggactaccttgaattccccaaatcgcttgttcataaaccacatccataccttgcccattctttttttttgagaccagggcttgctgtgttgcccaggctggattgcaatggcatgatcacagctcactgcagcttcaacctcctgggctcaagtgatcctcccatctcagcttcccaactagctgacactacaggcacgcacctccatgcttggctagtttgttaatatttttatagagatggggtctcagtatattgcccaggctggtcttgaactcttgcactcaagcaatcctcccacccctacctcccaaagtagcataagctactgcatctggccccattcttttacttgcgtactactaacttgcccatagcagaaagctctgaaatgttctggaattaggaacttcatatccctttattctctttattttttatttatttatttatttatttatttatttattgagataaggtttcactctgnnacccaggctggagtncagtggcccaattanagctcactgtancctctacctcctgggctaaagmaatcctcccatctcagccccttgagtanctgagactaaaggtgcacgccaccatgactggctttttttttttttagatggagtcttgctctgtcgccaggctggagtgcagtagtgcgatctctgctcactgcaacctccacctcccagattcaagcaattctcttgactcagcctcccaagtagctgggaccacaggtgcacgccaccatgctcagctaatttttgtacttttagtaatgacaggtttcaccatgttggccaggatggtctcgatctcttgacctcatgatccacccacatcagactcccaaagtgctaggattacaggcgtgagcnnnngcacctggcatttctttttttttaaaaaaagagacaaggtcttgcttgcccaggctgatctagaactcctgggctcaagcagtcctctcacctcagcatcccaaagtgctggaattgttggcctttattccctatacttcctattttgagccactaagcagtaaccattcaactaagatatctttgaaaatgactgctaccttatatcccttctcaccttaggcctctcttctgtggaaactctgaagccgaccagttgggcaaaatctttgagtaagtgaccaacatgggagaaaaagattttctattctgagtcctctttctgctgaacccaggatggcaactggctctgccatggggatgggaactggaggaccctcctgaccagagttctcctgtcccccacagcctgattgggctgcctccagaggatgactggcctcgagatgtatccctgccccgtggagcctttccccccagagggccccgcccagtgcagtcggtggtacctgagatggaggagtcgggagcacagctgctgctggtaactggagatggctgtgggcacagggaaagaaatagagactggggaaagaaatagagcagtatgcagggccctggccactgtggttaatgaaacttggttggtagatggtctgtagtttttattacagctgcaaatagccacccacagagaaggatatagaagagaacccatcctggctgggcacggtggctcacgcctgtaatcccagcactttgggaggccaaggtgggcgtatcacctgaggtcaggagttcgagaccagcctggccaacatggtgaaacctcgtctctactaaaagtacaaaaataagccgggggtggtggcacacgcctgtaatctcagctacttgggaggctgagataggagaatcacttcaactcaggaggcggaggttgcagtgagctgagatcataccattggcactccagcctgggtgatagagcgagactccgtctncaaaaaaaaaaaaaaagaaaaaagaagaaagctcatcccaggtattgttgtgggtggcagaagctgttttcttcatggttttctgacctttgcctctcccctcaggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaaccaaaagaaawaatggcccttttttttttttt

SEQ ID NO: 17 - protein, 438 aa, Homo sapiens
MLWLALGPFPAMENQVLVIRIKIPNSGAVDWTVHSGPQLLFRDVLDVIGQVLPEATTTAFEYEDEDGDRITVRSDEEMKAMLSYYYSTVMEQQVNGQLIEPLQIFPRACKPPGERNIHGLKVNTRAGPSQHSSPAVSDSLPSNSLKKSSAELKKILANGQMNEQDIRYRDTLGHGNGGTVYKAYHVPSGKILAVKVILLDITLELQKQIMSELEILYKCDSSYIIGFYGAFFVENRISICTEFMDGGSLDVYRKMPEHVLGRIAVAVVKGLTYLWSLKILHRDVKPSNMLVNTRGQVKLCDFGVSTQLVNSIAKTYVGTNAYMAPERISGEQYGIHSDVWSLGISFMEIQKNQGSLMPLQLLQCIVDEDSPVLPVGEFSEPFVHFITQCMRKQPKERPAPEELMGHPFIVQFNDGNAAVVSMWVCRALEERRSQQGPP

SEQ ID NO: 18 - DNA, 2083 bp, Homo sapiens
agcgttcgctcaactccagaaccttccgacctccgctagttcctgcgggcctttgcccgcttcccggtgcaccctccccgggagacacctcagacccccgacagcctgggcaggctcggtgcctgcgggtgcgttcctgatcacccctcccctcttccctccccctcatcctccattcccttgttttcaccctctgtcctctgcccgtcactccccttgtcacctcttggagccccctcctaaccagcggccagtgggtttcccataccccaggatgtgagcctctttaacctgtaatgctgtggctagcccttggcccctttcctgccatggagaaccaggtgctggtaattcgcatcaagatcccaaatagtggcgcggtggactggacagtgcactccgggccgcagttactcttcagggatgtgctggatgtgataggccaggttctgcctgaagcaacaactacagcatttgaatatgaagatgaagatggtgatcgaattacagtgagaagtgatgaggaaatgaaggcaatgctgtcatattattattccacagtaatggaacagcaagtaaatggacagttaatagagcctctgcagatatttccaagagcctgcaagcctcctggggaacggaacatacatggcctgaaggtgaatactcgggccggaccctctcaacacagcagcccagcagtctcagattcacttccaagcaatagcttaaagaagtcttctgctgaactgaaaaaaatactagccaatggccagatgaatgaacaagacatacgatatcgggacactcttggtcatggcaacggaggcacagtctacaaagcatatcatgtcccgagtgggaaaatattagctgtaaaggtcatactactagatattacactggaacttcagaagcaaattatgtctgaattggaaattctttataagtgcgattcatcatatatcattggattttatggagcattttttgtagaaaacaggatttcaatatgtacagaattcatggatgggggatctttggatgtatataggaaaatgccagaacatgtccttggaagaattgcagtagcagttgttaaaggccttacttatttgtggagtttaaagattttacatagagacgtgaagccctccaatatgctagtaaacacaagaggacaggttaagctgtgtgattttggagttagcactcagctggtgaattctatagccaagacgtatgttggaacaaatgcttatatggcgcctgaaaggatttcaggggagcagtatggaattcattctgatgtctggagcttaggaatctcttttatggagattcagaaaaaccagggatctttaatgcctctccagcttctgcagtgcattgttgatgaggattcgcccgtccttccagttggagagttctcggagccatttgtacatttcatcactcagtgtatgcgaaaacagccaaaagaaaggccagcacctgaagaattgatgggccacccgttcatcgtgcagttcaatgatggaaatgccgccgtggtgtccatgtgggtgtgccgggcgctggaggagaggcggagccagcaggggcccccgtgaggctgccgcagggcactgaaagcccaggaccagtaaccaaggagaacaacccacccgtcgcccttctccgtatgctgcctgcgccagaagagctttgctgggccctggcttccctgccctcgccttcacctctgtcagcaggtggccttgcctggggagccccatgtgtggcccaccccaccaggccatccccataccttctggtttgaaggcgctgacactggcagagaggtaaagggtggggcattgagaatggaggctcccagggtccctgcccacttctgttttcctaatgtttttctctataaagggtcaggcccgtcagcatcactgatgggaataaaagtattaatgctttgtgacagcctctgcctgaaaactggacagaaggacccagaggtgttctttcattttctctcttacctccaatctttcccctttcaagctacaggtaaaggctctaccaccat

SEQ ID NO: 19 - protein, 870 aa, Homo sapiens [sequence omitted]

SEQ ID NO: 20 - DNA, 3314 bp, Homo sapiens
  misc_feature (3228)..(3228): n is a, c, g, or t
catgaggagctgagcgtctcgggcgaggcgggctgacggcagcaccatgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggccggcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataaaaactatcgagaacagaggtcaaatgggctctctcattcttggagtgaaaggattccagacacaaaacatatttcagacatctgtgaaaatgggcgacctcgaagtaactcttggcaaggtaacctgggaggcaacaaaaagaaaatcagaggcaaaagatttagacctcggtctaattcaactgagactttatctcctgccaagtctccttcttcatcaactgggtctattgcctccagcagaaaatacccttacccaatgcctccacttcctgatgaggacaagaaagtgaaccgacaaagtgccaggctatgggagacatccatttaagatcaactgtttacatgtgatacatcgaaaactgtttacttcaacttttatagaaacccaggctcatggaatcactgcaaatctatctgctcttcagacaatacgaagaccctctgagatgctacagaggagaggaagcggagtttcacatctggttaccattttctttttgtcattggcttaggatttaactaaccatgaaaagaactactgaaatattacactataacatggaacaataaaggtactggtatgttaatggataatccgcatgacagataatatgtagaaatattcataaagttaactcacatgacccaaatgtagcaagtttcctaaggtacaatagtggattcagaacttgacgttctgaggcacatcctcactgtaaacagtaatgctatatgcatgaagcttctgtttattgttttccatatttaaggaaacaacatcccataatagaaatgagcatgcagggctaaggcatataggatttttctgcaggactttaaagctttgaaaggccaatatcccataggctaactttaaacatgtatttttatttttgntttgntttttacttttcatatttatattagcatacaaggacaattgnatatatgtaacatttttaaaattttaaaaaaaaaaaaaa

SEQ ID NO: 21 - protein, 824 aa, Homo sapiens
MRQSLLFLTSVVPFVLAPRPPDDPGFGPHQRLEKLDSLLSDYDILSLSNIQQHSVRKRDLQTSTHVETLLTFSALKRHFKLYLTSSTERFSQNFKVVVVDGKNESEYTAKWQDFFTGHVVGEPDSRVLAHIRDDDVIIRINTDGAEYNIEPLWRFVNDTKDKRMLVYKSEDIKNVSRLQSPKVCGYLKVDNEELLPKGLVDREPPEELVHRVKRRADPDPMKNTCKLLVVADHRFYRYMGRGEESTTTNYLIELIDRVDDIYRNTSWDNAGFKGYGIQIEQIRILKSPQEVKPGEKHYNMAKSYPNEEKDAWDVKMLLEQFSFDIAEEASKVCLAHLFTYQDFDMGTLGLAYVGSPRANSHGGVCPKAYYSPVGKKNIYLNSGLTSTKNYGKTILTKEADLVTTHELGHNFGAEHDPDGLAECAPNEDQGGKYVMYPIAVSGDHENNKMFSNCSKQSIYKTIESKAQECFQERSNKVCGNSRVDEGEECDPGIMYLNNDTCCNSDCTLKEGVQCSDRNSPCCKNCQFETAQKKCQEAINATCKGVSYCTGNSSECPPPGNAENDTVCLDLGKCKDGKCIPFCEREQQLESCACNETDNSCKVCCRDLSGRCVPYVDAEQKNLFLRKGKPCTVGFCDMNGKCEKRVQDVIERFWDFIDQLSINTFGKFLADNIVGSVLVFSLIFWIPFSILVHCVDKKLDKQYESLSLFHPSNVEMLSSMDSASVRIIKPFPAPQTPGRLQPAPVIPSAPAAPKLDHQRMDTIQEDPSTDSHMDEDGFEKDPFPNSSTAAKSFEDLTDHPVARSEKAASFKLQRQNRVNSKETEC

SEQ ID NO: 22 - DNA, 3523 bp, Homo sapiens
tcgagcctggcggtagaatcttcccagtaggcggcgcgggagggaaaagaggattgaggggctaggccgggcggatcccgtcctcccccgatgtgagcagttttccgaaaccccgtcaggcgaaggctgcccagagaggtggagtcggtagcggggccgggaacatgaggcagtctctcctattcctgaccagcgtggttcctttcgtgctggcgccgcgacctccggatgacccgggcttcggcccccaccagagactcgagaagcttgattctttgctctcagactacgatattctctctttatctaatatccagcagcattcggtaagaaaaagagatctacagacttcaacacatgtagaaacactactaactttttcagctttgaaaaggcattttaaattatacctgacatcaagtactgaacgtttttcacaaaatttcaaggtcgtggtggtggatggtaaaaacgaaagcgagtacactgcaaaatggcaggacttcttcactggacacgtggttggtgagcctgactctagggttctagcccacataagagatgatgatgttataatcagaatcaacacagatggggccgaatataacatagagccactttggagatttgttaatgataccaaagacaaaagaatgttagtttataaatctgaagatatcaagaatgtttcacgtttgcagtctccaaaagtgtgtggttatttaaaagtggataatgaagagttgctcccaaaagggttagtagacagagaaccacctgaagagcttgttcatcgagtgaaaagaagagctgacccagatcccatgaagaacacgtgtaaattattggtggtagcagatcatcgcttctacagatacatgggcagaggggaagagagtacaactacaaattacttaatagagctaattgacagagttgatgacatctatcggaacacttcatgggataatgcaggttttaaaggctatggaatacagatagagcagattcgcattctcaagtctccacaagaggtaaaacctggtgaaaagcactacaacatggcaaaaagttacccaaatgaagaaaaggatgcttgggatgtgaagatgttgctagagcaatttagctttgatatagctgaggaagcatctaaagtttgcttggcacaccttttcacataccaagattttgatatgggaactcttggattagcttatgttggctctcccagagcaaacagccatggaggtgtttgtccaaaggcttattatagcccagttgggaagaaaaatatctatttgaatagtggtttgacgagcacaaagaattatggtaaaaccatccttacaaaggaagctgacctggttacaactcatgaattgggacataattttggagcagaacatgatccggatggtctagcagaatgtgccccgaatgaggaccagggagggaaatatgtcatgtatcccatagctgtgagtggcgatcacgagaacaataagatgttttcaaactgcagtaaacaatcaatctataagaccattgaaagtaaggcccaggagtgttttcaagaacgcagcaataaagtttgtgggaactcgagggtggatgaaggagaagagtgtgatcctggcatcatgtatctgaacaacgacacctgctgcaacagcgactgcacgttgaaggaaggtgtccagtgcagtgacaggaacagtccttgctgtaaaaactgtcagtttgagactgcccagaagaagtgccaggaggcgattaatgctacttgcaaaggcgtgtcctactgcacaggtaatagcagtgagtgcccgcctccaggaaatgctgaaaatgacactgtttgcttggatcttggcaagtgtaaggatgggaaatgcatccctttctgcgagagggaacagcagctggagtcctgtgcatgtaatgaaactgacaactcctgcaaggtgtgctgcagggacctttctggccgctgtgtgccctatgtcgatgctgaacaaaagaacttatttttgaggaaaggaaagccctgtacagtaggattttgtgacatgaatggcaaatgtgagaaacgagtacaggatgtaattgaacgattttgggatttcattgaccagctgagcatcaatacttttggaaagtttttagcagacaacatcgttgggtctgtcctggttttctccttgatattttggattcctttcagcattcttgtccattgtgtggataagaaattggataaacagtatgaatctctgtctctgtttcaccccagtaacgtcgaaatgctgagcagcatggattctgcatcggttcgcattatcaaaccctttcctgcgccccagactccaggccgcctgcagcctgcccctgtgatcccttcggcgccagcagctccaaaactggaccaccagagaatggacaccatccaggaagaccccagcacagactcccatatggacgaggatgggtttgagaaggaccccttcccaaatagcagcacagctgccaagtcatttgaggatctcacggaccatccggtcgccagaagtgaaaaggctgcctcctttaaactgcagcgtcagaatcgtgttaacagcaaagaaacagagtgctaatttagttctcagctcttctgacttaagtgtgcaaaatatttttatagatttgacctacaaatcaatcacagcttgtattttgtgaagactgggaagtgacttagcagatgctggtcatgtgtttgaacttcctgcaggtaaacagttcttgtgtggtttggcccttctccttttgaaaaggtaaggtgaaagtgaatctacttattttgaggctttcaggttttagtttttaaaatatcttttgacctgtggtgcaaaagcagaaaatacagctggattgggttatgaatatttacgtttttgtaaattaatcttttatattgataacagcactgactagggaaatgatcagtttttttttatacactgtaatgaaccgctgaatatgaagcatttggcatttatttgtgagaaaagtggaatagttttttttttttttttttttttttgccttcaactaaaaacaaaggagataaatttagtatacattgtatctaaattgtgggtctatttctagttattacccagagtttttatgtagcagggaaaatatatatctaaatttagaaatcatttgggttaatatggctcttcataattctaagactaatgctcagaacctaaccactaccttacagtgagggctatacatggtagccagttgaatttatggaatctaccaactgtttagggccctgatttgctgggcagtttttctgtattttataagtatcttcatgtatccctgttactgatagggatacatgtcttagaaaattcactattggctgggagtggtggctcatgcctgtaatcccagcacttggagaggctgaggttgcgccactacactccagcctgggtgacagagtgagatctgcctc

SEQ ID NO: 23 - DNA, 1471 bp, Homo sapiens
gtggccgcgcaggaggacggagccctaaccgcaacccgcgccgcgccgcgccgatttgatttgtatccactgtcaccagcactgctcacttaggactttctggatccagacccaggcagcgcacactggactcttgaggaagaaggagactctaattttggattccttggtggaggaaaataaaacactctggtcttgccgccaacgatgcaagtgtgactgctggcgtcttcatgagctccagaggtcacagcacgctaccaaggactctcatggcccctcggatgatttccgagggagacataggaggcattgctcaaatcacctcctctctattcctgggcagaggcagtgtggcctccaatcggcacctcctccaggctcgtggcatcacctgcattgttaatgctaccattgagatccctaatttcaactggccccaatttgagtatgttaaagtgcctctggctgacatgccgcatgcccccattggactgtactttgacaccgtggctgacaagatccacagtgtgagcaggaagcacggggccaccttggtgcactgtgctgcaggggtgagccgctcagccacgctgtgtatcgcgtacctgatgaaattccacaacgtgtgcctgctggaggcatacaactgggtgaaagcccggcgacctgtcatcaggcccaacgtaggcttctggaggcaactgatagactacgagcgccagctctttgggaagtcgacagttaaaatggtacagacaccttatggcatagttcccgacgtctatgagaaggagtcccgacacctgatgccttactgggggatttagtgccactgaagcctgcgtcagcagcccgagcggggccggcatctgctccccgccgtctgctccctctccactctcttctcaaatggctgacttctggttctccctcaagtgttttttacactgggtgttcaaatttattttaagagatagggagggaggggacataaagggaatgcatacattgctagtcacatttttaaaattaacattttggaatagtgtttatggaaatctttagcttttaatcatttttaccaatttgaacagtttaataaactggttctgctctcttctgaatctcatgcctttggcaccttggtaggtgcaggaggagctcagtgcaaaaatcactttggggcctcattaaccctttagagacaagctttgccccaggctgcggaccagacagatgcttagggaaggttgataccagcttcagtctctactggattagccctactctttcctttcccctccattatttagtgactctgtaagtaagttaaatacacccttattatttagctgttaagtaactataatgaaatctgctgcaaaatctctcttggaatccatgtgcccaggattatattagcattatttttaataaatctatatgcttaaaaaaaaaaaaaaaaaaa

SEQ ID NO: 24 - protein, 689 aa, Homo sapiens
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKRDTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELHSLLEGLLQRDVNRRLGCLGRGAQEVKESPFFRSLDWQMVFLQRYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGL

SEQ ID NO: 25 - DNA, 3603 bp, Homo sapiens
ctcggcctcgggcgcggccgagcgccgcgcgagcaggagcggcggcggcggcggcggcggcgggaggaggcagcgccggcccaagatggcggacctggaggcggtgctggccgacgtgagctacctgatggccatggagaagagcaaggccacgccggccgcgcgcgccagcaagaagatactgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaagcgtgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacactccctgctggaggggttgctgcagagggatgtcaaccggagattgggctgcctgggccgaggggctcaggaggtgaaagagagcccctttttccgctccctggactggcagatggtcttcttgcagaggtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaacccctttctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctctgacccgcccacccgccttttataaacctctaatttattttgtcgaatttttattatttgttttcccgccaagcgaaaaggttttattttgtaattattgtgatttcccgtggccccagcctggcccagctcccccgggaggccccgcttgcctcggctcctgctgcaccaacccagccgctgcccggcgccctctgtcctgacttcaggggctgcccgctcccagtgtcttcctgtgggggaagagcacagccctcccgccccttccccgagggatgatgccacaccaagctgtgccaccctgggctctgtgggctgcacttgtgccatgggactgtgggtggcccatcccccctcaccaggggcaggcacagcacagggatccgacttgaattttcccactgcaccccctcctgctgcagaggggcaggccctgcactgtcctgctccacagtgttggcgagaggaggggcccgttgtctccctggccctcaaggctcccacagtgactcgggctcctgtgcccttattcaggaaaagcctctgtgtcactggctgcctccactcccacttccctgacactgcggggcttggctgagagagtggcattggcagcaggtgctgctaccctccctgctgtcccctcttgccccaacccccagcacccgggctcagggaccacagcaaggcacctgcaggttgggccatactggcctcgcctggcctgaggtctcgctgatgctgggctgggtgcgaccccatctgcccaggacggggccggccaggtgggcgggcagcacagcaaggaggctggctggggcctatcagtgtgccccccatcctggcccatcagtgtacccccgcccagactggccagccccacagcccacgtcctgtcagtgccgccgcctcgcccaccgcatgccccctgtgccagtgctctgcctgtgtgtgtgcactcgtgtcgcgccttctcccccccggggctgggttggcgcaccctcccctcccgtctactcattccccggggcgtttctttgccgatttttgaatgtgattttaaagagtgaaaaatgagactatgcgtttttataaaaaatggtgcctgattcggctgtctcagactctttttgtacctggtgaccccttttcagcttctgctgggctggggcctgatggggagggtctcggtggtaccaggtctcctccaccgccatggcttccaaggtggtctgctcgggcccaggccatcttccaggtggggtgaggcagtgggtcccacttcccctcctacccctcccagctgacagtcctctccacctagtggctgtccagtgcccattcctcaccttttcccggggaggagagagcagcttctgccacttcccaggtaagcaggaggaggtgccaacagtgttaggcctggcacagtgtctgggtgatcgggacct

SEQ ID NO: 26 - protein, 198 aa, Homo sapiens
MSSRGHSTLPRTLMAPRMISEGDIGGIAQITSSLFLGRGSVASNRHLLQARGITCIVNATIEIPNFNWPQFEYVKVPLADMPHAPIGLYFDTVADKIHSVSRKHGATLVHCAAGVSRSATLCIAYLMKFHNVCLLEAYNWVKARRPVIRPNVGFWRQLIDYERQLFGKSTVKMVQTPYGIVPDVYEKESRHLMPYWGI

SEQ ID NO: 27 - protein, 919 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNASFSLFSRNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGSAHRRAGHRERGGVLPGPGPAVYLSRPPACPQGLYELMLRCWSRESEQRPPFSQLHRFLAEDALNTV

SEQ ID NO: 28 - DNA, 3970 bp, Homo sapiens
ggcttaggaagtattaactgatctctgccctagttctcatgtgttaaatatggatagtaatagtatctaccttatgaagtgactgtgaagataaaattatggattctgtttaagggtttaggccagtgtctggcacaggggaagcattctaaaaatatagctgatgctgttaaacaatgactgttgttgttgttttactgttattatccccaaagcggcccattctgtctgttgctgtcagctatgactcagtcccctgattaacttacgcaccacccattttatcccctgcagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcgttgctgctctccaatccagcctaccgcctccttctggccacttacgcccgtccccctcgaggcccgggcccccccacacccgcctgggccaaacccaccaacacccaggcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccagcttctccttgttctccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggtcagctcaccgacgagcaggtcatcgagaacgcgggggagttcttccgggaccagggccggcagtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaa

SEQ ID NO: 29 - DNA, 17 bp, Artificial
  Primer
tttttttttttttttvn

SEQ ID NO: 30 - DNA, 21 bp, Artificial
  Primer
aagtacgagaagctggagacg

SEQ ID NO: 31 - DNA, 21 bp, Artificial
  PCR primer
aacatccttctggacgagcat

SEQ ID NO: 32 - DNA, 21 bp, Artificial
  Primer
aacgtacgcggaatacttcga

SEQ ID NO: 33 - DNA, 21 bp, Artificial
  Primer
aacttacgctgagtacttcga